(19) United States
(12) Patent Application Publication
(10) Pub. No.: US 2012/0178693 A1
(43) Pub. Date: Jul. 12, 2012

(54) Cofactors for Thrombin Activation of Factor VII and Uses Thereof

(75) Inventors: David Light, Somerville, MA (US); Maxine Bauzon, Hercules, CA (US); David Kiewlich, Alameda, CA (US); Terry Hermiston, Mill Valley, CA (US)

(73) Assignee: BAYER HEALTHCARE LLC, Tarrytown, NY (US)

(21) Appl. No.: 13/393,157

(22) PCT Filed: Aug. 28, 2010
(86) PCT No.: PCT/US2010/047066

§ 371 Date: Mar. 23, 2012

Related U.S. Application Data
(60) Provisional application No. 61/238,126, filed on Aug. 28, 2009.

(51) Int. Cl. A61K 38/17; C12N 15/62; A61P 7/04; C12N 5/10; C12N 5/16; C07K 19/00; C12N 15/63

(52) U.S. Cl. 514/14.7; 530/350; 530/381; 536/23.4; 435/320.1; 435/365; 435/352; 435/369; 435/353; 435/358; 435/346; 435/370; 435/354; 530/383

(57) ABSTRACT

The invention relates to fusion proteins that bind the enzyme thrombin and enhance the activation of the substrate Factor VII to the product Factor VIIa. The invention is also directed to polynucleotides, vectors, host cells, pharmaceutical compositions, and methods of treatment.

### REFERENCE TO RELATED APPLICATIONS

This application claims benefit of priority to U.S. Provisional Application 61/238,126 filed 28 Aug. 2010 which is hereby incorporated by reference.

### FIELD OF THE INVENTION

The invention describes the design and production of fusion proteins that are useful to treat patients with hemorrhages and bleeding disorders. These fusion proteins bind the enzyme thrombin and enhance the activation by thrombin of the substrate Factor VII (FVII) to the product Factor VIIa (FVIIa) (FIG. 1A). These fusion proteins act as soluble cofactors to increase formation of FVIIa at sites where thrombin is being generated during hemostasis. This increased FVIIa enhances thrombosis by both tissue factor (TF)-dependent and tissue factor (TF)-independent pathways. The fusion proteins consist of a thrombin binding domain, a linker, and a FVII binding domain with the following properties: (1) the thrombin binding domain binds thrombin at a site which does not interfere with the thrombin active site function, (2) the FVII binding domain binds FVII and allows it to be activated by thrombin, and (3) the linker domain allows the active site of bound thrombin to access and cleave the activation peptide of FVIIa.

### BACKGROUND OF THE INVENTION

The fusion proteins described in this invention act as soluble cofactors to enhance the activation of FVII at sites where thrombin is being generated by the coagulation cascade during thrombus formation (Butenas, et al., Biochemistry (Mosc) 67:3-12, 2002). These fusion proteins function in a similar manner as the cofactor thrombomodulin which binds thrombin and is a cofactor for the activation of protein C by thrombin (Esmon, Chest 124:26S-32S, 2003). However, in contrast to the thrombomodulin cofactor, the fusion proteins described in this invention act as cofactors for the enhanced activation of FVII, not protein C. Specific cleavage of FVII to FVIIa has been demonstrated and is known in the literature (Radcliffe, et al., J. Biol. Chem. 250:388-395, 1975; Butenas, et al., Biochemistry 35:1904-1910, 1996). However, the rate of activation by thrombin in the presence or absence of phospholipids (PCPS) is not considered to be sufficient to support large increases in FVIIa under physiological conditions by thrombin alone. Thrombin does not activate FVII as effectively as Factor Xa (FXa) on PCPS or as the complex of FVIIa and TF on PCPS (Butenas, et al., 1996; Yamamoto, et al., J. Biol. Chem. 267:19089-19094, 1992; Neuenschwander, et al., J. Biol. Chem. 268:21489-21492, 1993). When thrombin is bound to a cofactor, such as thrombomodulin, the rate at which it can cleave substrates that also bind to thrombomodulin is greatly enhanced. Important examples include the substrates protein C (Esmon, 2003), thrombin activatible fibrinolysis inhibitor, TAFI (Bajzar, et al., J. Biol. Chem. 271:16603-16608, 1996), and amphoterin or high mobility group box 1, HMGB1 (Ito, et al., Arterioscler. Throm. Vasc. Biol. 29:1825-1830, 2008). During these reactions, the anion-binding exosite I (ABE-I) of the enzyme thrombin binds to thrombomodulin via the C-loop of EGF4, EGF5, and EGF6 and this fragment of the extracellular domain of thrombomodulin is the minimal fragment needed to bind the enzyme thrombin. Molecules of thrombomodulin that have a chondroitin sulphate molecule added to the O-linked glycosylation domain are capable to bind two molecules of thrombin (Weisel, et al., J. Biol. Chem. 271:31485-31490, 1996) and are more effective cofactors for the activation of protein C by thrombin (Parkinson, et al., Biochem. J. 283:151-157, 1992; Ye, et al., J. Biol. Chem. 268:2373-2379, 1993).

The substrate, FVII can bind to one molecule of TF in a substrate-like manner during the auto-activation of FVII by the complex of FVIIa to a second molecule of TF (Neuenschwander, et al., J. Biol. Chem. 268:21489-21492, 1993). The x-ray crystal structure of FVIIa bound to TF is known (Banner, et al., Nature 380:41-46, 1996). TF is known to interact with the two EGF-like domains and the γ-carboxyglutamic acid (Gla) domain of FVIIa and FVII. The endothelial protein C receptor (EPCR) binds FVII and FVIIa with similar affinity (Rao, et al., Thromb Res. 122 Suppl 1:S3-6, 2008; Ghosh, et al., J. Biol. Chem. 282, 11849-11857, 2007) and this interaction is mediated by a Gla domain interaction with FVII (Preston, et al., J. Biol. Chem. 281:28850-28857, 2006). The cleavage site on the activation peptide of FVII, shown from the P4 to P4′ amino acid sites is: Pro₁₂Gln₁₃Gly₁₄Arg₁₅|Ile₁₆Val₁₇Gly₁₈Gly₁₉ (SEQ ID NO: 1), where the vertical link indicates the cleavage site. Based on over 140 thrombin cleavage sites annotated in the MEROPS the Peptidase Database (merops.sanger.ac.uk), this cleavage site on FVII is a consensus cleavage site for thrombin.

### SUMMARY OF THE INVENTION

The present application provides fusion proteins that include a thrombin binding domain, a linker, and a FVII binding domain with the following properties: (1) the thrombin binding domain binds thrombin at a site which does not interfere with the thrombin active site function, and (2) the FVII binding domain binds FVII and allows it to be activated by thrombin, and (3) the linker domain allows the active site of bound thrombin to access and cleave the activation peptide of FVIIa.

In one embodiment, the fusion proteins may comprise one or more thrombin binding domains. The thrombin binding domain may be the thrombomodulin thrombin binding domain, HCII thrombin binding domain, PAR1 thrombin binding domain, FVIII thrombin binding domain, OPN thrombin binding domain, HIR thrombin binding domain, FV thrombin binding domain, and FXI thrombin binding domain. For example, the fusion proteins may comprise one or more thrombin binding domains selected from SEQ ID NO: 28-30 and SEQ ID NO: 32-38. In another embodiment, the fusion proteins may comprise one or more FVII binding domains. The FVII binding domain may be the TF FVII binding domain or EPCR FVII binding domain. For example, the fusion proteins may comprise one or more FVII binding domains selected from SEQ ID NO: 27 and SEQ ID NO: 31.

In a further embodiment, the fusion proteins may comprise a linker For example, the fusion proteins may comprise a linker selected from SEQ ID NO: 2-19. In addition, the fusion proteins may comprise a site for chondroitin sulfate attachment (e.g., SEQ ID NO: 19). In another embodiment, the fusion protein may comprise a secretion signal. The secretion signal may be the secretion signal for TF, thrombomodulin, EPCR, kappa light chain, or FXI. For example, the fusion protein may comprise a secretion signal selected from SEQ ID NO: 20-26. In addition, the fusion protein may comprise a peptide tag (e.g., SEQ ID NO: 39 and 40) for detection or purification.

The fusion proteins of the present invention may comprise one or more thrombin binding domains, one or more FVII binding domains, a linker, and a secretion signal. For example, the fusion proteins may comprise one or more thrombin binding domains selected from SEQ ID NO: 28-30 and SEQ ID NO: 32-38, one or more FVII binding domains selected from SEQ ID NO: 27 and SEQ ID NO: 31, a linker selected from SEQ ID NO: 2-19, and a secretion signal selected from SEQ ID NO: 20-26. In one embodiment, the fusion proteins may be selected from SEQ ID NO: 41, 43, 45, 47, 49, and 51-84. In another embodiment, the fusion proteins may further comprise a peptide tag selected from SEQ ID NO: 39 and 40.

Additional thrombin binding sites may be added by including O-linked glycosylation sites (e.g., SEQ ID NO. 19) that result in the addition of chondroitin sulfate or similar anionic glycosaminoglycans. Examples of fusion proteins containing chondroitin sulfate sites are disclosed in SEQ ID NO: 51, 52, 55, and 56.

The present invention also includes polynucleotide sequences encoding the amino acid sequences of the fusion proteins, vectors, host cells, and methods of producing fusion proteins. In addition, the invention includes pharmaceutical compositions and methods of treatment.

### BRIEF DESCRIPTION OF THE FIGURES

FIG. 1 is a schematic of the function and design of a fusion protein. (A) Schematic representation of the recruitment of FVII and thrombin (Th) by soluble tissue factor (sTF) and thrombomodulin (TMcE56) derived regions of the fusion protein, respectively, and the subsequent cleavage and activation of FVII by thrombin. (B) Schematic representation of fusion protein constructs sTF-TMcE56-A (GSIGGGIS, SEQ ID NO: 2), sTF-TMcE56-B (GSIGGGGSGGGGSGGGGSGGGGSIS, SEQ ID NO: 3), and sTF-TMcE56-C (GSIGGGGSGGGGSGGGGSGGGGSGGGGSIS, SEQ ID NO. 4) constructs.

FIGS. 2A and B are Western blots stained with anti-human tissue factor (anti-hTF) antibody. Expression of fusion proteins in media of transfected 293 cells (probed with anti-hTF antibody).

FIG. 3 is a anti-TF ELISA. Quantification of fusion proteins in media (diluted 1:5) of transfected 293 cells using an anti-TF ELISA.

FIG. 4 demonstrates FVII activation by thrombin. FVII was incubated with increasing amounts of thrombin and the subsequent formation of active FVII was measured by monitoring the rate of hydrolysis of the chromogenic substrate Chromozym-tPA.

FIG. 5 illustrates CMK-treated FVII activation by thrombin. FVII was treated with a chloromethylketone (CMK) peptide inhibitor to inhibit activated proteases present in the substrate. CMK-FVII was incubated with increasing amounts of thrombin and the subsequent formation of active FVII was measured by monitoring the rate of hydrolysis of the chromogenic substrate Chromozym-tPA.

FIG. 6 illustrates FVII activation by thrombin with different linker lengths in the fusion protein.

### DESCRIPTION OF THE INVENTION

This invention describes the design and production of fusion proteins that are useful to treat patients with hemorrhages and bleeding disorders, including hemophilia A or Factor VIII (FVIII) deficiencies such as congenital hemophilia A (Sacchi, et al., Int. J. Clin. Lab. Res. 21:310-3, 1992), acquired hemophilia A (Huth-Kühne, et al., Haematologica. 94:459-61, 2009), and hemophilia A with FVIII inhibitors (Zhang, et al., Clin. Rev. Allergy Immunol. February 6., Epub, 2009), hemophilia B or Factor IX (FIX) deficiency (Kurachi, et al., Hematol. Oncol. Clin. North Am. 6:991-997, 1992; Lillicrap, Haemophilia 4:350-357, 1998), von Willebrand's disease (Castaman, et al., Haematologica. 88:94-108, 2003), Glanzmann disease, inherited coagulation disorders, inherited platelet disorders, hemorrhagic stroke, trauma, patients treated with heparin, aspirin, warfarin or other anticoagulant or antiplatelet drugs, and other bleeding diseases. These fusion proteins bind thrombin and enhance the activation of FVII to FVIIa by thrombin. These fusion proteins act as soluble cofactors to enhance the activation FVII at sites where thrombin is being generated during normal hemostasis. This increased FVII activation creates a local increase in FVIIa at the site where thrombin is formed. This increased FVIIa may further increase local thrombosis by both TF-dependent and -independent pathways. These fusion proteins consist of a thrombin binding domain, a linker and a FVII binding domain with the following functions: (1) the thrombin binding domain binds thrombin at a site which does not block or interfere with the thrombin active site, (2) a FVII binding domain which binds FVII and allows it to be activated by thrombin, and (3) a linker domain with a length and design that allows the active site of bound thrombin to access and cleave the activation peptide of FVII to generate FVIIa.

The thrombin enzyme binding domain may be derived from native or mutant forms of the following proteins or related proteins with the desired thrombin binding properties: thrombomodulin, the C-loop of EGF4 and the EGF5 and EGF6 loops of thrombomodulin, ABE-I peptide from heparin cofactor II, FVIII, Factor V (FV), PAR-1, osteopontin, or hirudin, the anion binding exosite II (ABE-II) of glycoprotein 1bα, the Apple 1 domain of Factor XI (FIX), antibodies that bind thrombin, or other non-antibody binding molecules that bind thrombin. The thrombin binding domain may be created by introducing sequences that are modified by post-translational modification including tyrosine sulfation and glycosylation. Glycosylation may be performed by appropriate cells or chemically to result in the attachment of ABE-II binding polysaccharides including heparin, chondroitin sulfate, and related polysaccharides. Finally, an ABE-I binding site and an ABE-II binding site may be combined in one thrombin binding domain to allow binding of more than one enzyme thrombin as the C-loop of EGF4, EGF5, or EGF6 and the O-linked glycosylation domain of thrombomodulin.

The FVII substrate binding domain may be derived from native or mutant forms of the TF, native or mutant forms of the N-terminal fibronectin-like domain of TF, native or mutant forms of endothelial protein C receptor (EPCR), FVII- or FVIIa-specific antibodies, and other non-antibody binding molecules that bind FVIIa or FVII. The linker domain must be of optimal length and structural design to allow interaction of the bound forms of thrombin and FVII.

The application provides a number of exemplary variants of fusion protein in which functional thrombin binding domains are derived from thrombin binding domains of human proteins. Additional thrombin binding sites may be added by including O-linked glycosylation sites that result in the addition of chondroitin sulfate or similar anionic glycosaminoglycans.

Due to the low molecular weight and compact structure, these fusion proteins may be administered by subcutaneous injection in order to allow convenient treatment of hemophilia A and hemophilia B. The current standard treatment of both diseases requires intravenous administration of plasma-derived or recombinant clotting factor.

The clearance and biodistribution of the fusion proteins described herein may be modified by post-translational modifications, including N-linked and O-linked glycosylation. These fusion proteins may comprise one or more glycosylation sites introduced, for example, by converting an endogenous O-linked glycosylation site to an N-linked glycosylation site. It has been reported that N-linked glycosylation sites are more likely to be sialylated than O-linked glycosylation sites and there is evidence that higher sialic acid content confers increased protein half-life. It is generally believed that the increased sialic acid content provided by additional N-linked glycosylation may be responsible for the increased half-life in blood (White, et al., Thromb. Haemost. 78:261-265, 1997).

### Production of Fusion Proteins

Amino acid sequence alteration may be accomplished by a variety of techniques such as, for example, by modifying the corresponding nucleic acid sequence by site-specific mutagenesis. Techniques for site-specific mutagenesis are well known in the art and are described in, for example, Zoller, et al., (DNA 3:479-488, 1984) or Horton, et al., (Gene 77:61-68, 1989, pp. 61-68). For example, a conservative substitution is recognized in the art as a substitution of one amino acid for another amino acid that has similar properties and include, for example, the changes of alanine to serine or arginine to lysine. Thus, using the nucleotide and amino acid sequences of the fusion proteins, one may introduce the alteration(s) of choice. Likewise, procedures for preparing a DNA construct using polymerase chain reaction using specific primers are well known to persons skilled in the art (see, e.g., PCR Protocols, 1990, Academic Press, San Diego, Calif., USA).

The nucleic acid construct encoding the fusion protein may also be prepared synthetically by established standard methods, for example, the phosphoramidite method described by Beaucage, et al., (Gene Amplif. Anal. 3:1-26, 1983). According to the phosphoamidite method, oligonucleotides are synthesized, for example, in an automatic DNA synthesizer, purified, annealed, ligated, and cloned in suitable vectors. The DNA sequences encoding the fusion protein polypeptides may also be prepared by polymerase chain reaction using specific primers, for example, as described in U.S. Pat. No. 4,683,202, or Saiki, et al., (Science 239:487-491, 1988). Furthermore, the nucleic acid construct may be of mixed synthetic and genomic, mixed synthetic and cDNA, or mixed genomic and cDNA origin prepared by ligating fragments of synthetic, genomic, or cDNA origin (as appropriate), corresponding to various parts of the entire nucleic acid construct, in accordance with standard techniques.

The DNA sequences encoding the fusion proteins may be inserted into a recombinant vector using recombinant DNA procedures. The choice of vector will often depend on the host cell into which the vector is to be introduced. The vector may be an autonomously replicating vector or an integrating vector. An autonomously replicating vector exists as an extrachromosomal entity and its replication is independent of chromosomal replication, for example, a plasmid. An integrating vector is a vector that integrates into the host cell genome and replicates together with the chromosome(s) into which it has been integrated.

The vector may be an expression vector in which the DNA sequence encoding the fusion protein is operably linked to additional segments required for transcription, translation, or processing of the DNA, such as promoters, terminators, and polyadenylation sites. In general, the expression vector may be derived from plasmid or viral DNA, or may contain elements of both. The term “operably linked” indicates that the segments are arranged so that they function in concert for their intended purposes, for example, transcription initiates in a promoter and proceeds through the DNA sequence coding for the polypeptide.

Expression vectors for use in expressing fusion proteins may comprise a promoter capable of directing the transcription of a cloned gene or cDNA. The promoter may be any DNA sequence that shows transcriptional activity in the host cell of choice and may be derived from genes encoding proteins either homologous or heterologous to the host cell. Examples of suitable promoters for directing the transcription of the DNA encoding the fusion protein in mammalian cells are, for example, the SV40 promoter (Subramani, et al., Mol. Cell Biol. 1:854-864, 1981), the MT-I (metallothionein gene) promoter (Palmiter, et al., Science 222:809-814, 1983), the CMV promoter (Boshart, et al., Cell 41:521-530, 1985), the myeloproliferative sarcoma virus (MPSV) LTR promoter (Lin, et al., Gene. 147:287-92, 1994), or the adenovirus 2 major late promoter (Kaufman, et al., Mol. Cell. Biol. 2:1304-1319, 1982).

The DNA sequences encoding the fusion protein may also, if necessary, be operably connected to a suitable terminator, such as the human growth hormone terminator (Palmiter, et al., Science 222:809-814, 1983) or TPI1 (Alber, et al., J. MoI. Appl. Gen. 1:419-434, 1982), or ADII3 (McKnight, et al., EMBO J. 4:2093-2099, 1985) terminators. The expression vectors may also contain a polyadenylation signal located downstream of the insertion site. Polyadenylation signals include the early or late polyadenylation signal from SV40, the polyadenylation signal from the adenovirus 5 EIb region, the human growth hormone gene terminator (DeNoto, et al., Nucl. Acids Res. 9:3719-3730, 1981), or the polyadenylation signal from the human TF gene or the human thrombomodulin gene. The expression vectors may also include enhancer sequences, such as the SV40 enhancer.

To direct the fusion protein into the secretory pathway of the host cells, either the native TF or the native thrombomodulin secretory signal sequences may be used. Alternatively, a secretory signal sequence (also known as a leader sequence, prepro sequence, or pre sequence) may be provided in the recombinant vector. The secretory signal sequence may be joined to the DNA sequences encoding the fusion protein in the correct reading frame. Secretory signal sequences are commonly positioned 5′ to the DNA sequence encoding the peptide. Exemplary signal sequences include, for example, the MPIF-1 signal sequence and the stanniocalcin signal sequence. Additional examples of secretion signals include SEQ ID NO: 20-26.

The procedures used to ligate the DNA sequences coding for the fusion protein polypeptides, the promoter, and optionally the terminator and/or secretory signal sequence and to insert them into suitable vectors containing the information necessary for replication, are well known to persons skilled in the art (see, e.g., Sambrook, et al., Molecular Cloning: A Laboratory Manual, Cold Spring Harbor, N.Y., 1989).

Methods of transfecting mammalian cells and expressing DNA sequences introduced into the cells are described in, for example, Kaufman, et al., (J. Mol. Biol. 159:601-621, 1982); Southern, et al., (J. Mol. Appl. Genet. 1:327-341, 1982); Loyter, et al., (Proc. Natl. Acad. Sci. USA 79:422-426, 1982); Wigler, et al., (Cell 14:725-731, 1978); Corsaro, et al., (Somatic Cell Genetics 7:603-616, 1981), Graham, et al., (Virology 52:456-467, 1973); and Neumann, et al., (EMBO J. 1:841-845, 1982). Cloned DNA sequences may be introduced into cultured mammalian cells by, for example, lipofection, DEAE-dextran-mediated transfection, microinjection, protoplast fusion, calcium phosphate precipitation, retroviral delivery, electroporation, sonoporation, laser irradiation, magnetofection, natural transformation, and biolistic transformation (see, e.g., Mehier-Humbert, et al., Adv. Drug Deliv. Rev. 57:733-753, 2005). To identify and select cells that express the exogenous DNA, a gene that confers a selectable phenotype (a selectable marker) is generally introduced into cells along with the gene or cDNA of interest. Selectable markers include, for example, genes that confer resistance to drugs such as neomycin, puromycin, hygromycin, and methotrexate. The selectable marker may be an amplifiable selectable marker, which permits the amplification of the marker and the exogenous DNA when the sequences are linked Exemplary amplifiable selectable markers include dihydrofolate reductase (DHFR) and adenosine deaminase. It is within the purview of one skilled in the art to choose suitable selectable markers (see, e.g., U.S. Pat. No. 5,238,820).

After cells have been transfected with DNA, they are grown in an appropriate growth medium to express the gene of interest. As used herein the term “appropriate growth medium” means a medium containing nutrients and other components required for the growth of cells and the expression of the active fusion protein.

Media generally include, for example, a carbon source, a nitrogen source, essential amino acids, essential sugars, vitamins, salts, phospholipids, protein; and growth factors may also be provided. Drug selection is then applied to select for the growth of cells that express the selectable marker in a stable fashion. For cells that have been transfected with an amplifiable selectable marker, the drug concentration may be increased to select for an increased copy number of the cloned sequences, thereby increasing expression levels. Clones of stably transfected cells are then screened for expression of the fusion protein.

Examples of mammalian cell lines for use in the present invention are the COS-1 (ATCC CRL 1650), baby hamster kidney (BHK), HKB11 (Cho, et al., J. Biomed. Sci, 9:631-638, 2002), and HEK-293 (ATCC CRL 1573; Graham, et al., J. Gen. Virol. 36:59-72, 1977) cell lines. In addition, a number of other cell lines may be used within the present invention, including rat IIep I (rat hepatoma; ATCC CRL 1600), rat IIep II (rat hepatoma; ATCC CRL 1548), TCMK-1 (ATCC CCL 139), IIep-G2 (ATCC HB 8065), NCTC 1469 (ATCC CCL 9.1), CHO-K-1 (ATCC CCL 61), and CHO-DUKX cells (Urlaub, et al., Proc. Natl. Acad. Sci. USA 77:4216-4220, 1980).

Fusion proteins may be recovered from cell culture medium and may then be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation)), extraction (see, e.g., Protein Purification, Janson and Lars Ryden, editors, VCH Publishers, New York, 1989), or various combinations thereof. In an exemplary embodiment, the proteins may be purified by affinity chromatography on an anti-TF or anti-thrombomodulin antibody column, or both. Additional purification may be achieved by conventional chemical purification means, such as high performance liquid chromatography. Other methods of purification are known in the art, and may be applied to the purification of the fusion proteins (see, e.g., Scopes, R., Protein Purification, Springer-Verlag, N.Y., 1982).

Generally, “purified” shall refer to a protein, polypeptide, or peptide composition that has been subjected to fractionation to remove various other components, and which substantially retains its expressed biological activity. Where the term “substantially purified” is used, this designation shall refer to a composition in which the protein, polypeptide, or peptide forms the major component of the composition, such as constituting about 50%, about 60%, about 70%, about 80%, about 90%, about 95%, about 99%, or more of the proteins in the composition.

Various methods for quantifying the degree of purification of a protein are known to those of skill in the art. These include, for example, determining the specific activity of an active fraction, or assessing the amount of polypeptides within a fraction by SDS/PAGE analysis. An exemplary method for assessing the purity of a fraction is to calculate the specific activity of the fraction, compare the activity to the specific activity of the initial extract, and to thus calculate the degree of purity, herein assessed by a “-fold purification number.” The actual units used to represent the amount of activity will, of course, be dependent upon the particular assay technique.

The fusion proteins may be recombinantly expressed in tissue culture cells and glycosylation may be the result of the normal post-translational cell functioning of the host cell, such as a mammalian cell. Glycosylation sites may be introduced, for example, by deleting one or more amino acid residues, substituting one or more endogenous amino acid residue with another amino acid(s), or adding one or more amino acid residues.

In one embodiment, the fusion proteins may also be glycosylated. Glycosylation of proteins is typically either N-linked or O-linked N-linked refers to the attachment of a carbohydrate moiety to the side chain of an asparagine residue. The tripeptide sequences Asn-X-Ser and Asn-X-Thr, where X is any amino acid except proline, are the recognition sequences for enzymatic attachment of the carbohydrate moiety to the Asn side chain. Thus, the presence of either of these tripeptide sequences in a protein creates a potential N-linked glycosylation site. An exemplary N-linked glycosylation site may be represented as follows X1-Asn-X2-X3-X4; where X1 is optionally Asp, Val, Glu, Gly, or Ile; X2 is any amino acid except Pro; X3 is Ser or Thr; and X4 is optionally Val, Glu, Gly, Gln, or Ile. Addition of N-linked glycosylation sites to a protein may be accomplished by altering the amino acid sequence such that one or more of the above-described tripeptide sequences is introduced.

O-linked glycosylation refers to the attachment of one of the sugars N-aceytlgalactosamine, galactose, or xylose to a hydroxyamino acid, most commonly to serine or threonine, although attachment to 5-hydroxyproline or 5-hydroxylysine is also possible. Addition of O-linked glycosylation sites to a fusion protein may be accomplished by altering the amino acid sequence such that one or more Ser or Thr residues are introduced.

A variety of methods have been proposed in the art to customize the glycosylation pattern of a protein (see, e.g., WO 99/22764; WO 98/58964; WO 99/54342; US Publication No. 2008/0050772; and U.S. Pat. No. 5,047,335). Essentially, many of the enzymes required for the in vitro glycosylation of polypeptides have been cloned and sequenced. In some instances, these enzymes have been used in vitro to add specific sugars to an incomplete glycan molecule on a polypeptide. In other instances, cells have been genetically engineered to express a combination of enzymes and desired polypeptides such that addition of a desired sugar moiety to an expressed polypeptide occurs within the cell.

The application provides, in part, fusion proteins with introduced glycosylation sites, wherein the carbohydrate chain attached to the glycosylation site may have a mammalian carbohydrate chain structure, that is, a mammalian glycosylation pattern. In some embodiments, the carbohydrate chain has a human glycosylation pattern. As used herein, a pattern of glycosylation refers to the representation of particular oligosaccharide structures within a given population of fusion protein polypeptides. Non-limiting examples of such patterns include the relative proportion of oligosaccharide chains that (i) have at least one sialic acid residue; (ii) lack any sialic acid residues (i.e., are neutral in charge); (iii) have at least one terminal galactose residue; (iv) have at least one terminal N-acetylgalactosamine residue; (v) have at least one “uncapped” antenna, that is, have at least one terminal galactose or N-acetylgalactosamine residue; or (vi) have at least one fucose linked alpha1->3 to an antennary N-acetylglucosamine residue.

The pattern of glycosylation may be determined using any method known in the art, including, without limitation: high-performance liquid chromatography (HPLC); capillary electrophoresis (CE); nuclear magnetic resonance (NMR); mass spectrometry (MS) using ionization techniques such as fast-atom bombardment, electrospray, or matrix-assisted laser desorption (MALDI); gas chromatography (GC); and treatment with exoglycosidases in conjunction with anion-exchange (AIE)-HPLC, size-exclusion chromatography (SEC), or MS (see, e.g., Weber et al., Anal. Biochem. 225:135-142, 1995; Klausen et al., J. Chromatog. 718:195-202, 1995; Morris et al., in Mass Spectrometry of Biological Materials, McEwen et al., eds., Marcel Dekker, (1990), pp 137-167; Conboy et al., Biol. Mass Spectrom. 21:397-407, 1992; Hellerqvist, Meth. Enzymol. 193:554-573, 1990; Sutton et al., Anal. Biochem. 218:34-46, 1994; Harvey et al., Organic Mass Spectrometry 29:753-766, 1994).

“Homology” refers to the degree of similarity between two protein or polynucleotide sequences. The correspondence between two sequences may be determined by techniques known in the art. For example, homology may be determined by a direct comparison of the sequence information of the polynucleotide or protein sequences. Usually, two sequences may be homologous if the sequences exhibit at least 75% sequence identity, 80% sequence identity, 85% sequence identity, 90% sequence identity, or 95% sequence identity.

Thus, the invention encompasses polynucleotides or protein having 75%, 80%, 85%, 90%, 95%, or greater sequence identity to the polynucleotide or protein sequences set forth in SEQ ID NOs: 41 to 84 or to combinations the protein sequences set forth in SEQ ID NOs: 2 to 40 that result in the formation of fusion proteins described herein.

To determine the percent homology of two protein sequences, or of two polynucleotide sequences, the sequences are aligned for optimal comparison purposes. For example, gaps may be introduced in the sequence of one protein or polynucleotide for optimal alignment with the other protein or polynucleotide. The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As used herein, amino acid or nucleic acid “homology” is equivalent to amino acid or nucleic acid “identity.” The percent homology between the two sequences is a function of the number of identical positions shared by the sequences, that is, the percent homology equals the number of identical positions/total number of positions times 100.

The invention also encompasses fusion proteins having a lower degree of identity, but having sufficient similarity so as to perform one or more of the same functions performed by the fusion proteins of the invention. Similarity is determined by conserved amino acid substitution. Such substitutions are those that substitute a given amino acid in a protein by another amino acid of like characteristics. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg and replacements among the aromatic residues Phe, Trp, and Tyr.

The single letter abbreviation for a particular amino acid, its corresponding amino acid, and three letter abbreviation are as follows: A, alanine (Ala); C, cysteine (Cys); D, aspartic acid (Asp); E, glutamic acid (Glu); F, phenylalanine (Phe); G, glycine (Gly); H, histidine (His); I, isoleucine (Ile); K, lysine (Lys); L, leucine (Leu); M, methionine (Met); N, asparagine (Asn); P, proline (Pro); Q, glutamine (Gln); R, arginine (Arg); S, serine (Ser); T, threonine (Thr); V, valine (Val); W, tryptophan (Trp); Y, tyrosine (Tyr); and norleucine (Nle).

Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M. Stockton Press, New York, 1991). Computer program methods to determine identity and similarity between two sequences include, but are not limited to, GCG program package (Devereux, et al., Nucleic Acids Res. 12:387, 1984), BLASTP, BLASTN, FASTA (Atschul, et al., J. Molec. Biol. 215:403, 1990).

A variant can differ in amino acid sequence by one or more substitutions, deletions, insertions, inversions, fusions, and truncations or a combination of any of these. Another useful variation is one that provides for a protease cleavage site in the linker that joins the thrombin binding domain and the factor VII binding domain. Variants containing the protease cleavage site may be utilized in vivo to the limit the extent of prothrombotic activity by the fusion protein.

In addition, a variation may provide a peptide tag or peptide expression tag that is incorporated the fusion protein. The peptide tag can be a FLAG tag, a c-myc tag, an E-tag, a 6× His tag, or similar peptide tag. The peptide tag may occur at the N-terminus, the C-terminus or elsewhere in the fusion protein. The peptide tag is useful both in vivo and in vitro for detection, purification, or identification of the fusion protein. It will be generally understood by one skilled it the art that the peptide tag sequence will usually be removed from the sequence used in the preparation or expression of the final drug substance.

### Pharmaceutical Compositions

Based on well known assays used to determine the efficacy for treatment of conditions identified above in mammals, and by comparison of these results with the results of known medicaments that are used to treat these conditions, the effective dosage of the fusion proteins of this invention may readily be determined for treatment of each desired indication. The amount of the active ingredient to be administered in the treatment of one of these conditions can vary widely according to such considerations as the particular polypeptide and dosage unit employed, the mode of administration, the period of treatment, the age and sex of the patient treated, and the nature and extent of the condition treated.

The application provides, in part, compositions comprising fusion proteins as described herein. The compositions may be suitable for in vivo administration and are pyrogen free. The compositions may also comprise a pharmaceutically acceptable carrier. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Supplementary active ingredients also may be incorporated into the compositions.

The compositions of the present invention include classic pharmaceutical preparations. Administration of these compositions according to the present invention may be via any common route. The pharmaceutical compositions may be introduced into the subject by any conventional method, for example, by intravenous, intradermal, intramuscular, subcutaneous, intramammary, intraperitoneal, intrathecal, retrobulbar, intrapulmonary, oral, sublingual, nasal, anal, vaginal, or transdermal delivery, or by surgical implantation at a particular site. The treatment may consist of a single dose or a plurality of doses over a period of time.

The active compounds may be prepared for administration as solutions of free base or pharmacologically acceptable salts in water, suitably mixed with a surfactant, such as hydroxypropylcellulose. Dispersions also may be prepared in glycerol, liquid polyethylene glycols, and mixtures thereof, and in oils. Under ordinary conditions of storage and use, these preparations contain a preservative to prevent the growth of microorganisms.

The pharmaceutical forms, suitable for injectable use, include sterile aqueous solutions or dispersions and sterile powders for the extemporaneous preparation of sterile injectable solutions or dispersions. The form should be sterile and should be fluid to the extent that easy syringability exists. It should be stable under the conditions of manufacture and storage and should be preserved against the contaminating action of microorganisms, such as bacteria and fungi. The carrier may be a solvent or dispersion medium containing, for example, water, ethanol, polyol (e.g., glycerol, propylene glycol, and liquid polyethylene glycol, and the like) sucrose, L-histidine, polysorbate 80, or suitable mixtures thereof, and vegetable oils. The proper fluidity may be maintained, for example, by the use of a coating, such as lecithin, by the maintenance of the required particle size in the case of dispersion, and by the use of surfactants. The prevention of the action of microorganisms may be brought about by various antibacterial an antifungal agents, for example, parabens, chlorobutanol, phenol, sorbic acid, thimerosal, and the like. The injectable compositions may include isotonic agents, for example, sugars or sodium chloride. Prolonged absorption of the injectable compositions may be brought about by the use in the compositions of agents delaying absorption, for example, aluminum monostearate and gelatin.

Sterile injectable solutions may be prepared by incorporating the active compounds (e.g., fusion protein) in the required amount in the appropriate solvent with various of the other ingredients enumerated above, as required, followed by filtered sterilization.

Generally, dispersions may be prepared by incorporating the various sterilized active ingredients into a sterile vehicle that contains the basic dispersion medium and the required other ingredients from those enumerated above. In the case of sterile powders for the preparation of sterile injectable solutions, methods of preparation include, for example, vacuum-drying and freeze-drying techniques that yield a powder of the active ingredient plus any additional desired ingredient from a previously sterile-filtered solution thereof.

Upon formulation, solutions may be administered in a manner compatible with the dosage formulation and in such amount as is therapeutically effective. “Therapeutically effective amount” is used herein to refer to the amount of a polypeptide that is needed to provide a desired level of the polypeptide in the bloodstream or in the target tissue. The precise amount will depend upon numerous factors, for example, the particular fusion protein polypeptide, the components and physical characteristics of the therapeutic composition, intended patient population, mode of delivery, individual patient considerations, and the like, and can readily be determined by one skilled in the art, based upon the information provided herein.

The formulations may be easily administered in a variety of dosage forms, such as injectable solutions, and the like. For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation may be determined by one of skill in the art depending on the route of administration and the desired dosage (see, e.g., Remington's Pharmaceutical Sciences, Mack Publishing Co., Easton, Pa., 20^{th} edition, 2000, incorporated herein by reference). Such formulations may influence the physical state, stability, rate of in vivo release, and rate of in vivo clearance of the administered agents. Depending on the route of administration, a suitable dose may be calculated according to body weight, body surface area, or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials. Exemplary dosing schedules include, without limitation, administration five times a day, four times a day, three times a day, twice daily, once daily, three times weekly, twice weekly, once weekly, twice monthly, once monthly, and any combination thereof.

Appropriate dosages may be ascertained through the use of established assays for determining blood clotting levels in conjunction with relevant dose response data. The final dosage regimen may be determined by the attending physician, considering factors that modify the action of drugs, for example, the drug's specific activity, severity of the damage, and the responsiveness of the patient, the age, condition, body weight, sex and diet of the patient, the severity of any infection, time of administration, and other clinical factors.

The composition may also include an antimicrobial agent for preventing or deterring microbial growth. Non-limiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant may be present in the composition as well. Antioxidants may be used to prevent oxidation, thereby preventing the deterioration of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant may be present as an excipient. Exemplary surfactants include: polysorbates such as Tween®-20 (polyoxyethylenesorbitan monolaurate) and Tween®-80 (polyoxyethylenesorbitan monooleate) and pluronics such as F68 and F88 (both of which are available from BASF, Mount Olive, N.J.); sorbitan esters; lipids such as phospholipids such as lecithin and other phosphatidylcholines, phosphatidylethanolamines, fatty acids and fatty esters; steroids such as cholesterol; and chelating agents such as EDTA, zinc and other such suitable cations.

Acids or bases may be present as an excipient in the composition. Non-limiting examples of acids that may be used include hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of any individual excipient in the composition may vary depending on the activity of the excipient and particular needs of the composition. Typically, the optimal amount of any individual excipient may be determined through routine experimentation, that is, by preparing compositions containing varying amounts of the excipient (ranging from low to high), examining the stability and other parameters, and then determining the range at which optimal performance is attained with no significant adverse effects. Generally, the excipient may be present in the composition in an amount of about 1% to about 99% by weight, from about 5% to about 98% by weight, from about 15 to about 95% by weight of the excipient, with concentrations less than 30% by weight. These foregoing pharmaceutical excipients along with other excipients are described in “Remington: The Science & Practice of Pharmacy,” 19 ed., Williams & Williams, (1995); the “Physician's Desk Reference,” 52 ed., Medical Economics, Montvale, N.J. (1998); and Kibbe, A. H., Handbook of Pharmaceutical Excipients, 3 Edition, American Pharmaceutical Association, Washington, D.C., 2000.

### Exemplary Uses

The fusion proteins or compositions comprising the fusion proteins described herein may be used to treat any hemorrhage or bleeding disorder associated with hemophilia A or FVIII deficiencies, such as congenital hemophilia A (Sacchi, et al., Int. J. Clin. Lab. Res. 21:310-3, 1992), acquired hemophilia A (Huth-Kühne, et al., Haematologica. 94:459-61, 2009), and hemophilia A with FVIII inhibitors (Zhang, et al., Clin. Rev. Allergy Immunol. February 6. [Epub], 2009), and other disorders such as hemophilia B or FIX deficiency (Kurachi, et al., Hematol. Oncol. Clin. North Am. 6:991-997, 1992; Lillicrap, Haemophilia 4:350-357, 1998), von Willebrand's disease (Castaman, et al., Haematologica. 88:94-108, 2003), Glanzmann disease, inherited coagulation disorders, inherited platelet disorders, hemorrhagic stroke, trauma, patients treated with heparin, aspirin, warfarin or other anticoagulant or antiplatelet drugs, and other bleeding diseases. Symptoms of such bleeding disorders include, for example, severe epistaxis, oral mucosal bleeding, hemarthrosis, hematoma, persistent hematuria, gastrointestinal bleeding, retroperitoneal bleeding, tongue/retropharyngeal bleeding, intracranial bleeding, and trauma-associated bleeding.

The fusion proteins and compositions of the present invention may be used for prophylactic applications. In some embodiments, fusion proteins may be administered to a subject susceptible to or otherwise at risk of a disease state or injury to enhance the subject's own coagulative capability. Such an amount may be defined to be a “prophylactically effective dose.” Administration of the fusion protein polypeptides for prophylaxis includes situations where a patient suffering from hemorrhage or bleeding disorder is about to undergo surgery and the polypeptide is administered between one to four hours prior to surgery. In addition, the polypeptides are suited for use as a prophylactic against uncontrolled bleeding, optionally in patients not suffering from hemophilia. Thus, for example, the polypeptide may be administered to a patient at risk for uncontrolled bleeding prior to surgery.

The fusion proteins, materials, compositions, and methods described herein are intended to be representative examples of the invention, and it will be understood that the scope of the invention is not limited by the scope of the examples. Those skilled in the art will recognize that the invention may be practiced with variations on the disclosed polypeptides, materials, compositions and methods, and such variations are regarded as within the ambit of the invention.

The following examples are presented to illustrate the invention described herein, but should not be construed as limiting the scope of the invention in any way.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1

### Design of Fusion Proteins

The spatial orientation of the enzyme, thrombin and the substrate, FVII is modeled to be similar to the spatial orientation of thrombin and protein C in a model based on the x-ray crystal structure of thrombin and thrombomodulin (Fuentes-Prior, et al., Nature 404:518-25, 2000). The linker domain may either link the C-terminus of a FVII binding domain such as soluble TF, to the N-terminus of a thrombin binding domain such as soluble thrombomodulin, or link the C-terminus of a thrombin binding domain to the N-terminus of a FVII binding domain. In either case, the linker must be of sufficient length to allow the correct spatial orientation of enzyme and substrate.

The fusion proteins may comprise one or more of the following linker sequences:

(SEQ ID NO: 2)
GSIGGGIS,
(SEQ ID NO: 3)
GSIGGGGSGGGGSGGGGSGGGGSIS,
(SEQ ID NO. 4)
GSIGGGGSGGGGSGGGGSGGGGSGGGGSIS,
(SEQ ID NO. 5)
GSIGSGGGGSGGGGSGGGGSGGGGSGGGIS,
(SEQ ID NO. 6)
GSIGSGGGGSGGGGSGGGGSGGIS,
(SEQ ID NO. 7)
GGGGSGGGGS,
(SEQ ID NO. 8)
GGGGSGGGGSGGGGS,
(SEQ ID NO. 9)
GGGGSGGGGSGGGGSGGGGS,
(SEQ ID NO. 10)
GGGGSGGGGSGGGGSGGGGSGGGGS,
(SEQ ID NO. 11)
GGGGSGGGGSGGGGSGGGGSGGGGSGGGGS,
(SEQ ID NO. 12)
GGGGSGGGGSGGGGSGGGGSGGGGSGGGGSGGGGS,
(SEQ ID NO. 13)
GGGGSGGGGSPAPAPGGGGSGGGGSGGGGS,
(SEQ ID NO. 14)
GGGGSGGGGSGGGGSPAPAPGGGGSGGGGS,
(SEQ ID NO. 15)
GGGGSPAPAPGGGGSGGGGSPAPAPGGGGS,
(SEQ ID NO. 16)
GSGGSGSGGSGSGGSGSGGSGSGGSGSGGS,
(SEQ ID NO. 17)
GSGGSGSGGSGGPAPAPGGSGSGGSGSGGS,
(SEQ ID NO. 18)
GGGGSGGGGAEAAAKEAAAKAGGGSGGGGS,
(SEQ ID NO. 19)
DSGKVDGGDSGSGEPPPSPTPGSTLTPPAVGLVHS,
(SEQ ID NO. 93)
GGGGS,
and
(SEQ ID NO. 94)
GGGGSPAPAPGGGGSGGGGS.

The fusion protein may further comprise a secretion signal. The secretion signal may be the secretion signal for TF (SEQ ID NO: 20 and 21), thrombomodulin (SEQ ID NO: 22 and 23), EPCR (SEQ ID NO: 24), kappa light chain (SEQ ID NO: 25), or FXI (SEQ ID NO: 26):

(SEQ ID NO: 20)
METPAWPRVPRPGTAVARTLLLGWVFAQVAGA,
(SEQ ID NO: 21)
METPAWPRVPRPETAVARTLLLGWVFAQVAGA,
(SEQ ID NO: 22)
MLGVLVLGALALAGLVFP,
(SEQ ID NO: 23)
MLGVLVLGALALAGLGFP,
(SEQ ID NO: 24)
MLTTLLPILLLSGWA,
(SEQ ID NO: 25)
METDTLLLWVLLLWVPGSTGDAA,
and
(SEQ ID NO: 26)
MIFLYQVVHFILFTSVSG.

The fusion proteins of present invention may comprise one or more thrombin binding domains. The thrombin binding domain may be the thrombomodulin thrombin binding domain (SEQ ID NO: 28-30), HCII thrombin binding domain (SEQ ID NO: 32), PAR1 thrombin binding domain (SEQ ID NO: 33), FVIII thrombin binding domain (SEQ ID NO: 34), OPN thrombin binding domain (SEQ ID NO: 35), HIR thrombin binding domain (SEQ ID NO: 36), FV thrombin binding domain (SEQ ID NO: 37), and FXI thrombin binding domain (SEQ ID NO: 38). The fusion proteins may also comprise one or more FVII binding domains. The FVII binding domain may be the TF FVII binding domain (SEQ ID NO: 27) or EPCR FVII binding domain (SEQ ID NO: 31). For example, the fusion proteins may comprise one or more of the following sequences:

(SEQ ID NO: 27)
SGTTNTVAAYNLTWKSTNFKTILEWEPKPVNQVYTVQISTKSGDWKSKCF
YTTDTECDLTDEIVKDVKQTYLARVFSYPAGNVESTGSAGEPLYENSPEF
TPYLETNLGQPTIQSFEQVGTKVNVTVEDERTLVRRNNTFLSLRDVFGKD
LIYTLYYWKSSSSGKKTAKTNTNEFLIDVDKGENYCFSVQAVIPSRTVNR
KSTDSPVECMGQEKGEFRE,
(SEQ ID NO: 28)
VCAEGFAPIPGEPHRCQLFCNQTACPADCDPNTQASCECPEGYILDDGFI
CTDIDECENGGFCSGVCHNLPGTFECICGPDSALAGQIGTDC,
(SEQ ID NO: 29)
AVCAEGFAPIPHEPHRCQMFCNQTACPADCDPNTQASCECPEGYILDDGF
ICTDIDECENGGFCSGVCHNLPGTFECICGPDSALVRHIGTDCDSGKVDG
GD,
(SEQ ID NO: 30)
AVCAEGFAPIPHEPHRCQMFCNQTACPADCDPNTQASCECPEGYILDDGF
ICTDIDECENGGFCSGVCHNLPGTFECICGPDSALVRHIGTDC,
(SEQ ID NO: 31)
FCSQDASDGLQRLHMLQISYFRDPYHVWYQGNASLGGHLTHVLEGPDTNT
TIIQLQPLQEPESWARTQSGLQSYLLQFHGLVRLVHQERTLAFPLTIRCF
LGCELPPEGSRAHVFFEVAVNGSSFVSFRPERALWQADTQVTSGVVTFTL
QQLNAYNRTRYELREFLEDTCVQYVQKHISAENTKGSQTSRSYTS,
(SEQ ID NO: 32)
PEGEEDDDYLDLEKIFSEDDDYIDI,
(SEQ ID NO: 33)
NDKYEPFWEDEEKNESGLTEY,
(SEQ ID NO: 34)
NTGDYYEDSYEDISAYLLSKNNAIEPRSFS,
(SEQ ID NO: 35)
DIQYPDATDEDITSHMESEE,
(SEQ ID NO: 36)
NNGDFEEIPEEYLQ,
(SEQ ID NO: 37)
PDDDEDSYEIFEPPESTVMATRKMHDRLEPEDEESDADYDYQNRLAAALG
IRSFRN,
and
(SEQ ID NO: 38)
ECVTQLLKDTCFEGGDITTVFTPSAKYCQVVCTYHPRCLLFTFTAESPSE
DPTRWFTCVLKDSVTETLPRVNRTAAISGYSFKQCSHQISA.

The fusion proteins may also comprise one of the following tag sequences:

(SEQ ID NO: 39)
AAAGAPVPYPDPLEPRAA
and
(SEQ ID NO: 40)
AAADYKDDDDK.

Examples of fusion proteins of the invention are shown below. The fusion proteins may also include a peptide tag (e.g., SEQ ID NO: 39 or 40) for ease of detection and purification.

sTF-TMcE56-A:
(SEQ ID NO: 41)
METPAWPRVPRPGTAVARTLLLGWVFAQVAGASGTTNTVAAYNLTWKSTNFKTILEWEP
KPVNQVYTVQISTKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRRNNTFLSLRDVFG
KDLIYTLYYWKSSSSGKKTAKTNTNEFLIDVDKGENYCFSVQAVIPSRTVNRKSTDSPVEC
MGQEKGEFREGSIGGGISVCAEGFAPIPGEPHRCQLFCNQTACPADCDPNTQASCECPEGY
ILDDGFICTDIDECENGGFCSGVCHNLPGTFECICGPDSALAGQIGTDC
sTF-TMcE56-B:
(SEQ ID NO: 43)
METPAWPRVPRPGTAVARTLLLGWVFAQVAGASGTTNTVAAYNLTWKSTNFKTILEWEP
KPVNQVYTVQISTKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRRNNTFLSLRDVFG
KDLIYTLYYWKSSSSGKKTAKTNTNEFLIDVDKGENYCFSVQAVIPSRTVNRKSTDSPVEC
MGQEKGEFREGSIGGGGSGGGGSGGGGSGGGGSISVCAEGFAPIPGEPHRCQLFCNQTAC
PADCDPNTQASCECPEGYILDDGFICTDIDECENGGFCSGVCHNLPGTFECICGPDSALAGQ
IGTDC
sTF-TMcE56-C:
(SEQ ID NO: 45)
METPAWPRVPRPGTAVARTLLLGWVFAQVAGASGTTNTVAAYNLTWKSTNFKTILEWEP
KPVNQVYTVQISTKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRRNNTFLSLRDVFG
KDLIYTLYYWKSSSSGKKTAKTNTNEFLIDVDKGENYCFSVQAVIPSRTVNRKSTDSPVEC
MGQEKGEFREGSIGGGGSGGGGSGGGGSGGGGSGGGGSISVCAEGFAPIPGEPHRCQLFC
NQTACPADCDPNTQASCECPEGYILDDGFICTDIDECENGGFCSGVCHNLPGTFECICGPDS
ALAGQIGTDC
sTF-TMcE56-D:
(SEQ ID NO: 47)
METPAWPRVPRPETAVARTLLLGWVFAQVAGASGTTNTVAAYNLTWKSTNFKTILEWEP
KPVNQVYTVQISTKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRRNNTFLSLRDVFG
KDLIYTLYYWKSSSSGKKTAKTNTNEFLIDVDKGENYCFSVQAVIPSRTVNRKSTDSPVEC
MGQEKGEFREGSIGSGGGGSGGGGSGGGGSGGGGSGGGISVCAEGFAPIPHEPHRCQMFC
NQTACPADCDPNTQASCECPEGYILDDGFICTDIDECENGGFCSGVCHNLPGTFECICGPDS
ALVRHIGTDC
TMcE56-sTF:
(SEQ ID NO: 49)
MLGVLVLGALALAGLVFPAVCAEGFAPIPHEPHRCQMFCNQTACPADCDPNTQASCECPE
GYILDDGFICTDIDECENGGFCSGVCHNLPGTFECICGPDSALVRHIGTDCDSGKVDGGDG
SIGSGGGGSGGGGSGGGGSGGISSGTTNTVAAYNLTWKSTNFKTILEWEPKPVNQVYTVQ
ISTKSGDWKSKCFYTTDTECDLTDEIVKDVKQTYLARVFSYPAGNVESTGSAGEPLYENSP
EFTPYLETNLGQPTIQSFEQVGTKVNVTVEDERTLVRRNNTFLSLRDVFGKDLIYTLYYWK
SSSSGKKTAKTNTNEFLIDVDKGENYCFSVQAVIPSRTVNRKSTDSPVECMGQEKGEFRE
sTF-TMcE56-OlinkCS:
(SEQ ID NO: 51)
METPAWPRVPRPETAVARTLLLGWVFAQVAGASGTTNTVAAYNLTWKSTNFKTILEWEP
KPVNQVYTVQISTKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRRNNTFLSLRDVFG
KDLIYTLYYWKSSSSGKKTAKTNTNEFLIDVDKGENYCFSVQAVIPSRTVNRKSTDSPVEC
MGQEKGEFREGSIGSGGGGSGGGGSGGGGSGGGGSGGGISVCAEGFAPIPHEPHRCQMFC
NQTACPADCDPNTQASCECPEGYILDDGFICTDIDECENGGFCSGVCHNLPGTFECICGPDS
ALVRHIGTDCDSGKVDGGDSGSGEPPPSPTPGSTLTPPAVGLVHS
TMcE56-OlinkCS-sTF:
(SEQ ID NO: 52)
MLGVLVLGALALAGLVFPAVCAEGFAPIPHEPHRCQMFCNQTACPADCDPNTQASCECPE
GYILDDGFICTDIDECENGGFCSGVCHNLPGTFECICGPDSALVRHIGTDCDSGKVDGGDS
GSGEPPPSPTPGSTLTPPAVGLVHSSGTTNTVAAYNLTWKSTNFKTILEWEPKPVNQVYTV
QISTKSGDWKSKCFYTTDTECDLTDEIVKDVKQTYLARVFSYPAGNVESTGSAGEPLYEN
SPEFTPYLETNLGQPTIQSFEQVGTKVNVTVEDERTLVRRNNTFLSLRDVFGKDLIYTLYY
WKSSSSGKKTAKTNTNEFLIDVDKGENYCFSVQAVIPSRTVNRKSTDSPVECMGQEKGEF
RE
sEPCR-TMcE56:
(SEQ ID NO: 53)
MLTTLLPILLLSGWAFCSQDASDGLQRLHMLQISYFRDPYHVWYQGNASLGGHLTHVLE
GPDTNTTIIQLQPLQEPESWARTQSGLQSYLLQFHGLVRLVHQERTLAFPLTIRCFLGCELP
PEGSRAHVFFEVAVNGSSFVSFRPERALWQADTQVTSGVVTFTLQQLNAYNRTRYELREF
LEDTCVQYVQKHISAENTKGSQTSRSYTSGSIGSGGGGSGGGGSGGGGSGGGGSGGGISV
CAEGFAPIPHEPHRCQMFCNQTACPADCDPNTQASCECPEGYILDDGFICTDIDECENGGF
CSGVCHNLPGTFECICGPDSALVRHIGTDC
TMcE56-sEPCR:
(SEQ ID NO: 54)
MLGVLVLGALALAGLVFPAVCAEGFAPIPHEPHRCQMFCNQTACPADCDPNTQASCECPE
GYILDDGFICTDIDECENGGFCSGVCHNLPGTFECICGPDSALVRHIGTDCDSGKVDGGDG
SIGSGGGGSGGGGSGGGGSGGISFCSQDASDGLQRLHMLQISYFRDPYHVWYQGNASLG
GHLTHVLEGPDTNTTIIQLQPLQEPESWARTQSGLQSYLLQFHGLVRLVHQERTLAFPLTIR
CFLGCELPPEGSRAHVFFEVAVNGSSFVSFRPERALWQADTQVTSGVVTFTLQQLNAYNR
TRYELREFLEDTCVQYVQKHISAENTKGSQTSRSYTS
sEPCR-TMcE56-OlinkCS:
(SEQ ID NO: 55)
MLTTLLPILLLSGWAFCSQDASDGLQRLHMLQISYFRDPYHVWYQGNASLGGHLTHVLE
GPDTNTTIIQLQPLQEPESWARTQSGLQSYLLQFHGLVRLVHQERTLAFPLTIRCFLGCELP
PEGSRAHVFFEVAVNGSSFVSFRPERALWQADTQVTSGVVTFTLQQLNAYNRTRYELREF
LEDTCVQYVQKHISAENTKGSQTSRSYTSGSIGSGGGGSGGGGSGGGGSGGGGSGGGISV
CAEGFAPIPHEPHRCQMFCNQTACPADCDPNTQASCECPEGYILDDGFICTDIDECENGGF
CSGVCHNLPGTFECICGPDSALVRHIGTDCDSGKVDGGDSGSGEPPPSPTPGSTLTPPAVGL
VHS
TMcE56-OlinkCS-sEPCR:
(SEQ ID NO: 56)
MLGVLVLGALALAGLVFPAVCAEGFAPIPHEPHRCQMFCNQTACPADCDPNTQASCECPE
GYILDDGFICTDIDECENGGFCSGVCHNLPGTFECICGPDSALVRHIGTDCDSGKVDGGDS
GSGEPPPSPTPGSTLTPPAVGLVHSFCSQDASDGLQRLHMLQISYFRDPYHVWYQGNASL
GGHLTHVLEGPDTNTTIIQLQPLQEPESWARTQSGLQSYLLQFHGLVRLVHQERTLAFPLTI
RCFLGCELPPEGSRAHVFFEVAVNGSSFVSFRPERALWQADTQVTSGVVTFTLQQLNAYN
RTRYELREFLEDTCVQYVQKHISAENTKGSQTSRSYTS
HCIIABE-sTF:
(SEQ ID NO: 57)
METPAWPRVPRPETAVARTLLLGWVFAQVAGAPEGEEDDDYLDLEKIFSEDDDYIDIGSI
GSGGGGSGGGGSGGGGSGGGGSGGGISSGTTNTVAAYNLTWKSTNFKTILEWEPKPVNQ
VYTVQISTKSGDWKSKCFYTTDTECDLTDEIVKDVKQTYLARVFSYPAGNVESTGSAGEP
LYENSPEFTPYLETNLGQPTIQSFEQVGTKVNVTVEDERTLVRRNNTFLSLRDVFGKDLIY
TLYYWKSSSSGKKTAKTNTNEFLIDVDKGENYCFSVQAVIPSRTVNRKSTDSPVECMGQE
KGEFRE
sTF-HCIIABE:
(SEQ ID NO: 58)
METPAWPRVPRPETAVARTLLLGWVFAQVAGASGTTNTVAAYNLTWKSTNFKTILEWEP
KPVNQVYTVQISTKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRRNNTFLSLRDVFG
KDLIYTLYYWKSSSSGKKTAKTNTNEFLIDVDKGENYCFSVQAVIPSRTVNRKSTDSPVEC
MGQEKGEFREGSIGSGGGGSGGGGSGGGGSGGGGSGGGISPEGEEDDDYLDLEKIFSEDD
DYIDI
PAR1ABE-sTF:
(SEQ ID NO: 59)
METPAWPRVPRPETAVARTLLLGWVFAQVAGANDKYEPFWEDEEKNESGLTEYGSIGSG
GGGSGGGGSGGGGSGGGGSGGGISSGTTNTVAAYNLTWKSTNFKTILEWEPKPVNQVYT
VQISTKSGDWKSKCFYTTDTECDLTDEIVKDVKQTYLARVFSYPAGNVESTGSAGEPLYE
NSPEFTPYLETNLGQPTIQSFEQVGTKVNVTVEDERTLVRRNNTFLSLRDVFGKDLIYTLY
YWKSSSSGKKTAKTNTNEFLIDVDKGENYCFSVQAVIPSRTVNRKSTDSPVECMGQEKGE
FRE
sTF-PAR1ABE:
(SEQ ID NO: 60)
METPAWPRVPRPETAVARTLLLGWVFAQVAGASGTTNTVAAYNLTWKSTNFKTILEWEP
KPVNQVYTVQISTKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRRNNTFLSLRDVFG
KDLIYTLYYWKSSSSGKKTAKTNTNEFLIDVDKGENYCFSVQAVIPSRTVNRKSTDSPVEC
MGQEKGEFREGSIGSGGGGSGGGGSGGGGSGGGGSGGGISNDKYEPFWEDEEKNESGLT
EY
FVIIIABE-sTF:
(SEQ ID NO: 61)
METPAWPRVPRPETAVARTLLLGWVFAQVAGANTGDYYEDSYEDISAYLLSKNNAIEPRS
FSGSIGSGGGGSGGGGSGGGGSGGGGSGGGISSGTTNTVAAYNLTWKSTNFKTILEWEPK
PVNQVYTVQISTKSGDWKSKCFYTTDTECDLTDEIVKDVKQTYLARVFSYPAGNVESTGS
AGEPLYENSPEFTPYLETNLGQPTIQSFEQVGTKVNVTVEDERTLVRRNNTFLSLRDVFGK
DLIYTLYYWKSSSSGKKTAKTNTNEFLIDVDKGENYCFSVQAVIPSRTVNRKSTDSPVECM
GQEKGEFRE
sTF-FVIIIABE:
(SEQ ID NO: 62)
METPAWPRVPRPETAVARTLLLGWVFAQVAGASGTTNTVAAYNLTWKSTNFKTILEWEP
KPVNQVYTVQISTKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRRNNTFLSLRDVFG
KDLIYTLYYWKSSSSGKKTAKTNTNEFLIDVDKGENYCFSVQAVIPSRTVNRKSTDSPVEC
MGQEKGEFREGSIGSGGGGSGGGGSGGGGSGGGGSGGGISNTGDYYEDSYEDISAYLLSK
NNAIEPRSFS
OPNABE-sTF:
(SEQ ID NO: 63)
METPAWPRVPRPETAVARTLLLGWVFAQVAGADIQYPDATDEDITSHMESEEGSIGSGGG
GSGGGGSGGGGSGGGGSGGGISSGTTNTVAAYNLTWKSTNFKTILEWEPKPVNQVYTVQI
STKSGDWKSKCFYTTDTECDLTDEIVKDVKQTYLARVFSYPAGNVESTGSAGEPLYENSP
EFTPYLETNLGQPTIQSFEQVGTKVNVTVEDERTLVRRNNTFLSLRDVFGKDLIYTLYYWK
SSSSGKKTAKTNTNEFLIDVDKGENYCFSVQAVIPSRTVNRKSTDSPVECMGQEKGEFRE
sTF-OPNABE:
(SEQ ID NO: 64)
METPAWPRVPRPETAVARTLLLGWVFAQVAGASGTTNTVAAYNLTWKSTNFKTILEWEP
KPVNQVYTVQISTKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRRNNTFLSLRDVFG
KDLIYTLYYWKSSSSGKKTAKTNTNEFLIDVDKGENYCFSVQAVIPSRTVNRKSTDSPVEC
MGQEKGEFREGSIGSGGGGSGGGGSGGGGSGGGGSGGGISDIQYPDATDEDITSHMESEE
HIRABE-sTF :
(SEQ ID NO: 65)
METPAWPRVPRPETAVARTLLLGWVFAQVAGANNGDFEEIPEEYLQGSIGSGGGGSGGG
GSGGGGSGGGGSGGGISSGTTNTVAAYNLTWKSTNFKTILEWEPKPVNQVYTVQISTKSG
DWKSKCFYTTDTECDLTDEIVKDVKQTYLARVFSYPAGNVESTGSAGEPLYENSPEFTPY
LETNLGQPTIQSFEQVGTKVNVTVEDERTLVRRNNTFLSLRDVFGKDLIYTLYYWKSSSSG
KKTAKTNTNEFLIDVDKGENYCFSVQAVIPSRTVNRKSTDSPVECMGQEKGEFRE
sTF-HIRABE:
(SEQ ID NO: 66)
METPAWPRVPRPETAVARTLLLGWVFAQVAGASGTTNTVAAYNLTWKSTNFKTILEWEP
KPVNQVYTVQISTKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRRNNTFLSLRDVFG
KDLIYTLYYWKSSSSGKKTAKTNTNEFLIDVDKGENYCFSVQAVIPSRTVNRKSTDSPVEC
MGQEKGEFREGSIGSGGGGSGGGGSGGGGSGGGGSGGGISNNGDFEEIPEEYLQ
FVABE-sTF:
(SEQ ID NO: 67)
METPAWPRVPRPETAVARTLLLGWVFAQVAGAPDDDEDSYEIFEPPESTVMATRKMHDR
LEPEDEESDADYDYQNRLAAALGIRSFRNGSIGSGGGGSGGGGSGGGGSGGGGSGGGISS
GTTNTVAAYNLTWKSTNFKTILEWEPKPVNQVYTVQISTKSGDWKSKCFYTTDTECDLT
DEIVKDVKQTYLARVFSYPAGNVESTGSAGEPLYENSPEFTPYLETNLGQPTIQSFEQVGT
KVNVTVEDERTLVRRNNTFLSLRDVFGKDLIYTLYYWKSSSSGKKTAKTNTNEFLIDVDK
GENYCFSVQAVIPSRTVNRKSTDSPVECMGQEKGEFRE
sTF-FVABE:
(SEQ ID NO: 68)
METPAWPRVPRPETAVARTLLLGWVFAQVAGASGTTNTVAAYNLTWKSTNFKTILEWEP
KPVNQVYTVQISTKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRRNNTFLSLRDVFG
KDLIYTLYYWKSSSSGKKTAKTNTNEFLIDVDKGENYCFSVQAVIPSRTVNRKSTDSPVEC
MGQEKGEFREGSIGSGGGGSGGGGSGGGGSGGGGSGGGISPDDDEDSYEIFEPPESTVMA
TRKMHDRLEPEDEESDADYDYQNRLAAALGIRSFRN
Apple1-sTF:
(SEQ ID NO: 69)
MIFLYQVVHFILFTSVSGECVTQLLKDTCFEGGDITTVFTPSAKYCQVVCTYHPRCLLFTFT
AESPSEDPTRWFTCVLKDSVTETLPRVNRTAAISGYSFKQCSHQISAGSIGSGGGGSGGGG
SGGGGSGGGGSGGGISSGTTNTVAAYNLTWKSTNFKTILEWEPKPVNQVYTVQISTKSGD
WKSKCFYTTDTECDLTDEIVKDVKQTYLARVFSYPAGNVESTGSAGEPLYENSPEFTPYLE
TNLGQPTIQSFEQVGTKVNVTVEDERTLVRRNNTFLSLRDVFGKDLIYTLYYWKSSSSGK
KTAKTNTNEFLIDVDKGENYCFSVQAVIPSRTVNRKSTDSPVECMGQEKGEFRE
sTF-Apple1:
(SEQ ID NO: 70)
METPAWPRVPRPETAVARTLLLGWVFAQVAGASGTTNTVAAYNLTWKSTNFKTILEWEP
KPVNQVYTVQISTKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRRNNTFLSLRDVFG
KDLIYTLYYWKSSSSGKKTAKTNTNEFLIDVDKGENYCFSVQAVIPSRTVNRKSTDSPVEC
MGQEKGEFREGSIGSGGGGSGGGGSGGGGSGGGGSGGGISECVTQLLKDTCFEGGDITTV
FTPSAKYCQVVCTYHPRCLLFTFTAESPSEDPTRWFTCVLKDSVTETLPRVNRTAAISGYSF
KQCSHQISA
HCIIABE-sEPCR:
(SEQ ID NO: 71)
METPAWPRVPRPETAVARTLLLGWVFAQVAGAPEGEEDDDYLDLEKIFSEDDDYIDIGSI
GSGGGGSGGGGSGGGGSGGGGSGGGISFCSQDASDGLQRLHMLQISYFRDPYHVWYQGN
ASLGGHLTHVLEGPDTNTTIIQLQPLQEPESWARTQSGLQSYLLQFHGLVRLVHQERTLAF
PLTIRCFLGCELPPEGSRAHVFFEVAVNGSSFVSFRPERALWQADTQVTSGVVTFTLQQLN
AYNRTRYELREFLEDTCVQYVQKHISAENTKGSQTSRSYTS
sEPCR-HCIIABE:
(SEQ ID NO: 72)
MLTTLLPILLLSGWAFCSQDASDGLQRLHMLQISYFRDPYHVWYQGNASLGGHLTHVLE
GPDTNTTIIQLQPLQEPESWARTQSGLQSYLLQFHGLVRLVHQERTLAFPLTIRCFLGCELP
PEGSRAHVFFEVAVNGSSFVSFRPERALWQADTQVTSGVVTFTLQQLNAYNRTRYELREF
LEDTCVQYVQKHISAENTKGSQTSRSYTSGSIGSGGGGSGGGGSGGGGSGGGGSGGGISPE
GEEDDDYLDLEKIFSEDDDYIDI
PAR1-sEPCR:
(SEQ ID NO: 73)
METPAWPRVPRPETAVARTLLLGWVFAQVAGANDKYEPFWEDEEKNESGLTEYGSIGSG
GGGSGGGGSGGGGSGGGGSGGGISFCSQDASDGLQRLHMLQISYFRDPYHVWYQGNASL
GGHLTHVLEGPDTNTTIIQLQPLQEPESWARTQSGLQSYLLQFHGLVRLVHQERTLAFPLTI
RCFLGCELPPEGSRAHVFFEVAVNGSSFVSFRPERALWQADTQVTSGVVTFTLQQLNAYN
RTRYELREFLEDTCVQYVQKHISAENTKGSQTSRSYTS
sEPCR-PAR1:
(SEQ ID NO: 74)
MLTTLLPILLLSGWAFCSQDASDGLQRLHMLQISYFRDPYHVWYQGNASLGGHLTHVLE
GPDTNTTIIQLQPLQEPESWARTQSGLQSYLLQFHGLVRLVHQERTLAFPLTIRCFLGCELP
PEGSRAHVFFEVAVNGSSFVSFRPERALWQADTQVTSGVVTFTLQQLNAYNRTRYELREF
LEDTCVQYVQKHISAENTKGSQTSRSYTSGSIGSGGGGSGGGGSGGGGSGGGGSGGGISN
DKYEPFWEDEEKNESGLTEY
FVIIIABE-sEPCR:
(SEQ ID NO: 75)
METPAWPRVPRPETAVARTLLLGWVFAQVAGANTGDYYEDSYEDISAYLLSKNNAIEPRS
FSGSIGSGGGGSGGGGSGGGGSGGGGSGGGISFCSQDASDGLQRLHMLQISYFRDPYHVW
YQGNASLGGHLTHVLEGPDTNTTIIQLQPLQEPESWARTQSGLQSYLLQFHGLVRLVHQE
RTLAFPLTIRCFLGCELPPEGSRAHVFFEVAVNGSSFVSFRPERALWQADTQVTSGVVTFT
LQQLNAYNRTRYELREFLEDTCVQYVQKHISAENTKGSQTSRSYTS
sEPCR-FVIIIABE:
(SEQ ID NO: 76)
MLTTLLPILLLSGWAFCSQDASDGLQRLHMLQISYFRDPYHVWYQGNASLGGHLTHVLE
GPDTNTTIIQLQPLQEPESWARTQSGLQSYLLQFHGLVRLVHQERTLAFPLTIRCFLGCELP
PEGSRAHVFFEVAVNGSSFVSFRPERALWQADTQVTSGVVTFTLQQLNAYNRTRYELREF
LEDTCVQYVQKHISAENTKGSQTSRSYTSGSIGSGGGGSGGGGSGGGGSGGGGSGGGISN
TGDYYEDSYEDISAYLLSKNNAIEPRSFS
OPN-sEPCR:
(SEQ ID NO: 77)
METPAWPRVPRPETAVARTLLLGWVFAQVAGADIQYPDATDEDITSHMESEEGSIGSGGG
GSGGGGSGGGGSGGGGSGGGISFCSQDASDGLQRLHMLQISYFRDPYHVWYQGNASLGG
HLTHVLEGPDTNTTIIQLQPLQEPESWARTQSGLQSYLLQFHGLVRLVHQERTLAFPLTIRC
FLGCELPPEGSRAHVFFEVAVNGSSFVSFRPERALWQADTQVTSGVVTFTLQQLNAYNRT
RYELREFLEDTCVQYVQKHISAENTKGSQTSRSYTS
sEPCR-OPN:
(SEQ ID NO: 78)
MLTTLLPILLLSGWAFCSQDASDGLQRLHMLQISYFRDPYHVWYQGNASLGGHLTHVLE
GPDTNTTIIQLQPLQEPESWARTQSGLQSYLLQFHGLVRLVHQERTLAFPLTIRCFLGCELP
PEGSRAHVFFEVAVNGSSFVSFRPERALWQADTQVTSGVVTFTLQQLNAYNRTRYELREF
LEDTCVQYVQKHISAENTKGSQTSRSYTSGSIGSGGGGSGGGGSGGGGSGGGGSGGGISDI
QYPDATDEDITSHMESEE
HIR-sEPCR:
(SEQ ID NO: 79)
METPAWPRVPRPETAVARTLLLGWVFAQVAGANNGDFEEIPEEYLQGSIGSGGGGSGGG
GSGGGGSGGGGSGGGISFCSQDASDGLQRLHMLQISYFRDPYHVWYQGNASLGGHLTHV
LEGPDTNTTIIQLQPLQEPESWARTQSGLQSYLLQFHGLVRLVHQERTLAFPLTIRCFLGCE
LPPEGSRAHVFFEVAVNGSSFVSFRPERALWQADTQVTSGVVTFTLQQLNAYNRTRYELR
EFLEDTCVQYVQKHISAENTKGSQTSRSYTS
sEPCR-HIR:
(SEQ ID NO: 80)
MLTTLLPILLLSGWAFCSQDASDGLQRLHMLQISYFRDPYHVWYQGNASLGGHLTHVLE
GPDTNTTIIQLQPLQEPESWARTQSGLQSYLLQFHGLVRLVHQERTLAFPLTIRCFLGCELP
PEGSRAHVFFEVAVNGSSFVSFRPERALWQADTQVTSGVVTFTLQQLNAYNRTRYELREF
LEDTCVQYVQKHISAENTKGSQTSRSYTSGSIGSGGGGSGGGGSGGGGSGGGGSGGGISN
NGDFEEIPEEYLQ
FVABE-sEPCR:
(SEQ ID NO: 81)
METPAWPRVPRPETAVARTLLLGWVFAQVAGAPDDDEDSYEIFEPPESTVMATRKMHDR
LEPEDEESDADYDYQNRLAAALGIRSFRNGSIGSGGGGSGGGGSGGGGSGGGGSGGGISF
CSQDASDGLQRLHMLQISYFRDPYHVWYQGNASLGGHLTHVLEGPDTNTTIIQLQPLQEP
ESWARTQSGLQSYLLQFHGLVRLVHQERTLAFPLTIRCFLGCELPPEGSRAHVFFEVAVNG
SSFVSFRPERALWQADTQVTSGVVTFTLQQLNAYNRTRYELREFLEDTCVQYVQKHISAE
NTKGSQTSRSYTS
sEPCR-FVABE:
(SEQ ID NO: 82)
MLTTLLPILLLSGWAFCSQDASDGLQRLHMLQISYFRDPYHVWYQGNASLGGHLTHVLE
GPDTNTTIIQLQPLQEPESWARTQSGLQSYLLQFHGLVRLVHQERTLAFPLTIRCFLGCELP
PEGSRAHVFFEVAVNGSSFVSFRPERALWQADTQVTSGVVTFTLQQLNAYNRTRYELREF
LEDTCVQYVQKHISAENTKGSQTSRSYTSGSIGSGGGGSGGGGSGGGGSGGGGSGGGISP
DDDEDSYEIFEPPESTVMATRKMHDRLEPEDEESDADYDYQNRLAAALGIRSFRN
Apple1-sEPCR:
(SEQ ID NO: 83)
MIFLYQVVHFILFTSVSGECVTQLLKDTCFEGGDITTVFTPSAKYCQVVCTYHPRCLLFTFT
AESPSEDPTRWFTCVLKDSVTETLPRVNRTAAISGYSFKQCSHQISAGSIGSGGGGSGGGG
SGGGGSGGGGSGGGISFCSQDASDGLQRLHMLQISYFRDPYHVWYQGNASLGGHLTHVL
EGPDTNTTIIQLQPLQEPESWARTQSGLQSYLLQFHGLVRLVHQERTLAFPLTIRCFLGCEL
PPEGSRAHVFFEVAVNGSSFVSFRPERALWQADTQVTSGVVTFTLQQLNAYNRTRYELRE
FLEDTCVQYVQKHISAENTKGSQTSRSYTS
sEPCR-Apple1:
(SEQ ID NO: 84)
MLTTLLPILLLSGWAFCSQDASDGLQRLHMLQISYFRDPYHVWYQGNASLGGHLTHVLE
GPDTNTTIIQLQPLQEPESWARTQSGLQSYLLQFHGLVRLVHQERTLAFPLTIRCFLGCELP
PEGSRAHVFFEVAVNGSSFVSFRPERALWQADTQVTSGVVTFTLQQLNAYNRTRYELREF
LEDTCVQYVQKHISAENTKGSQTSRSYTSGSIGSGGGGSGGGGSGGGGSGGGGSGGGISE
CVTQLLKDTCFEGGDITTVFTPSAKYCQVVCTYHPRCLLFTFTAESPSEDPTRWFTCVLKD
SVTETLPRVNRTAAISGYSFKQCSHQISA.

### Example 2

### Cloning and Expression of Fusion Proteins

The DNA fragment encoding soluble tissue factor (sTF) was amplified by PCR from pMISC133 using the following primers:

(SEQ ID NO: 85)
5′GCGCCCAAGCTTGCGATGGAGACCCCTGCCTGGCCCCGGG-3′
and
(SEQ ID NO: 86)
5′GACGGATATCCCGCCCCCAATCGATCCTTCTCTGAATTCCCCTTTCTC
CTGGCCC-3′.

The DNA fragment encoding soluble thrombomodulin domain including all or part of EGF4, EGF5, EGF6, and the E-tag (TMcE56-etag) was amplified by PCR from pKM115.5 using the following primers:

(SEQ ID NO: 87)
5′GGCGGGATATCCGTCTGCGCCGAGGGCTTCGCGCCCATTCCC-3′
and
(SEQ ID NO: 88)
5′GCCGCTCGAGCGGTCATGCGGCACGCGGTTCCAGCGGATCCG-3.

Both fragments were subcloned into pCR2.1-Topo (Invitrogen, Carlsbad, Calif.) and the DNA sequence was verified. The sTF and TMe56-etag fragments were then subcloned into pCMV-Gluc via a three point ligation using the XhoI and HindIII sites. The resulting construct is designated sTF-TMcE56(A). The sTF-TMcE56(A) (SEQ ID NO: 41) plasmid was then transfected into INV110 (dam-) competent cells (Invitrogen, Carlsbad, Calif.) and digested with ClaI and EcoRV. The following linker oligo pairs were annealed and cloned into the prepared ClaI/EcoRV digested vector:

(SEQ ID NO: 89)
B5′CGATTGGCGGTGGTGGCTCCGGTGGCGGTGGTAGTGGCGGTGGTGGC
TCCGGCGGTGGTGGCTCGAT-3′,
(SEQ ID NO: 90)
B5′ATCGAGCCACCACCGCCGGAGCCACCACCGCCACTACCACCGCCACC
GGAGCCACCACCGCCAAT-3′,
(SEQ ID NO: 91)
C5′CGATTGGCGGTGGTGGCTCCGGCGGTGGTGGCAGCGGTGGCGGTGGT
AGTGGCGGTGGTGGCTCCGGCGGTGGTGGCTCGAT-3′,
and
(SEQ ID NO: 92)
C5′ATCGAGCCACCACCGCCGGAGCCACCACCGCCACTACCACCGCCACC
GCTGCCACCACCGCCGGAGCCACCACCGCCAAT-3′.

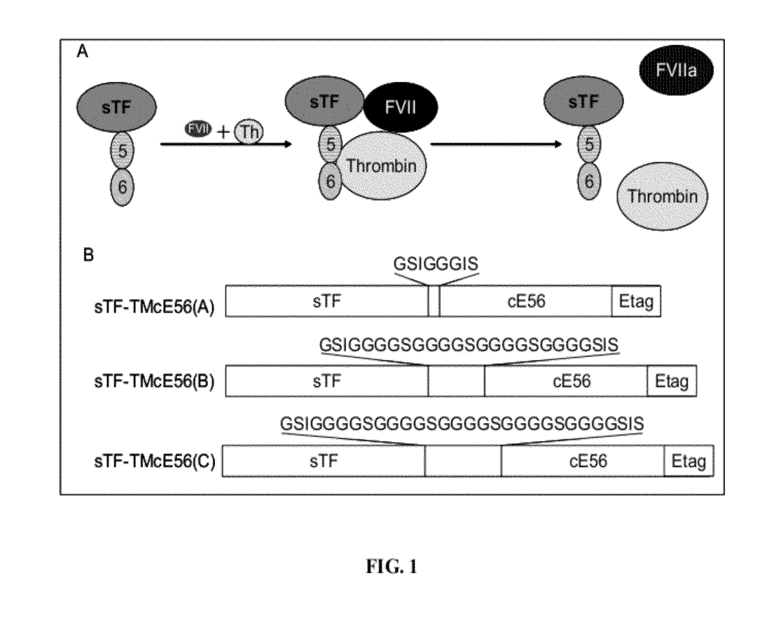

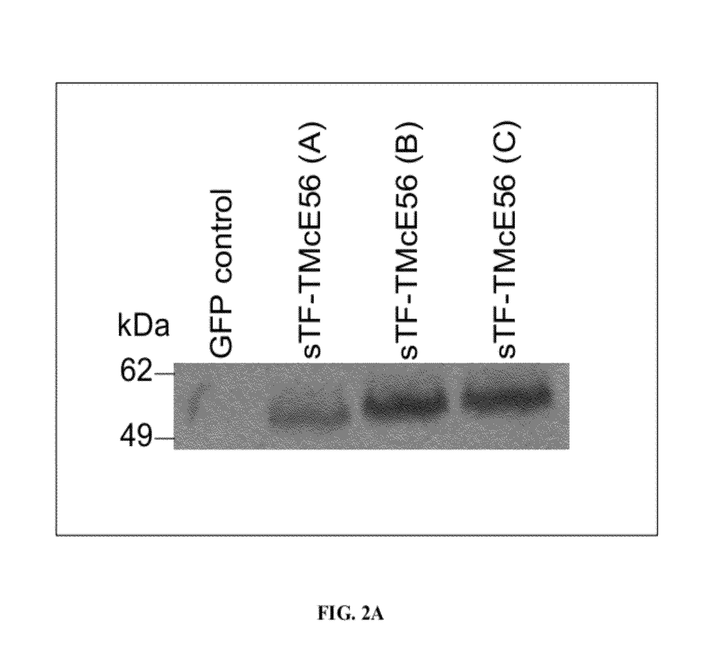

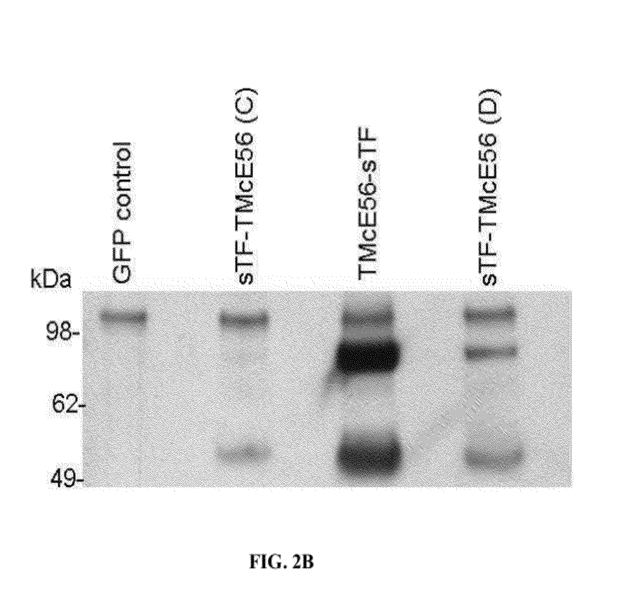

The resulting constructs were designated sTF-TMcE56(B) (SEQ ID NO: 43) and sTF-TMcE56(C) (SEQ ID NO: 45) (FIG. 1B). The sTF-TMcE56(D) (SEQ ID NO: 47) and TMcE56-sTF (SEQ ID NO: 49) inserts were synthesized and subcloned into the pCMV vector using the XhoI/HindIII sites. Fusion constructs sTF-TMcE56 (A), (B), and (C) and pEGFPNI as a control were transfected into 293 cells using FuGENE® 6 (Roche, Indianapolis, Ind.). Four days post transfection, the media from transfected cells were collected and subjected to SDS-PAGE gel electrophoresis and Western analysis using an anti-human tissue factor antibody (American Diagnostica, Stamford, Conn.). The expression of additional fusion proteins sTF-TMcE56(D) and TMcE56-sTF was tested in a similar manner. The results are shown in FIGS. 2A and 2B. FIG. 2A: Lane 1—GFP control is a negative control sample (cells transfected with a control vector expressing GFP (green fluorescent protein)); Lane 2—sTF-TMcE56(A); Lane 3—sTF-TMcE56(B); and Lane 4—sTF-TMcE56(C). FIG. 2B: Lane 1—GFP control; Lane 2—sTF-TMcE56(C); Lane 3—TMcE56-sTF; and Lane 4—sTF-TMcE56(D).

### Example 3

### Quantitation of Fusion Proteins Containing Tissue Factor by ELISA

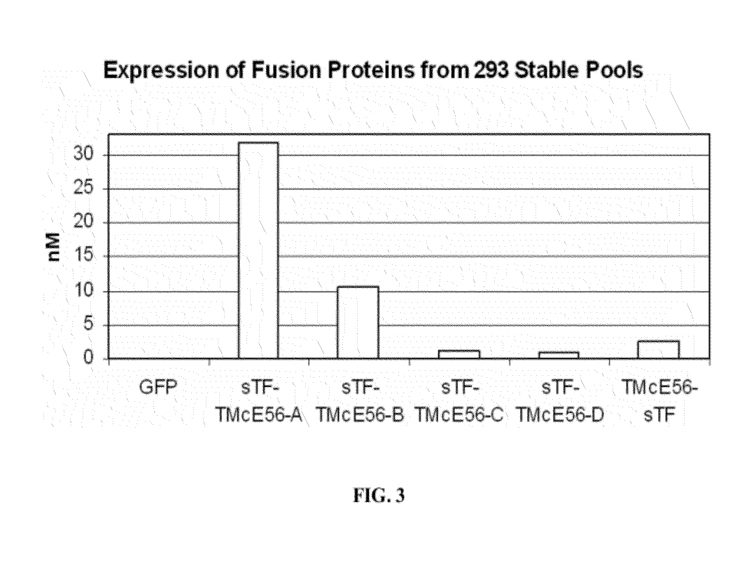

Expression levels were quantified using an anti-TF ELISA. Fusion constructs sTF-TMcE56 (A), (B), (C), and (D) and TMcE56-sTF, and pEGFPNI control were transfected into 293 cells using FuGENE® 6 (Roche, Indianapolis, Ind.). Four days post transfection, the media from transfected cells were collected and used for TF quantitation using the IMUBIND® Tissue Factor ELISA (American Diagnostica, Stamford, Conn.). The samples were diluted 1:000 except sTF-TMcE56 (A) which was diluted 1:2000). The expression level of the fusion proteins varies from 1 to >30 nM, depending on the construct, based on TF immunoreactivity. The results are shown in FIG. 3. Lane 1—GFP is a negative control; Lane 2—sTF-TMcE56-A; Lane 3—sTF-TMcE56-B; Lane 4—sTF-TMcE56-C; Lane 5—sTF-TMcE56-D; and Lane 6—TMcE56-sTF.

### Example 4

### Enzymatic Assay of Factor VII Activation

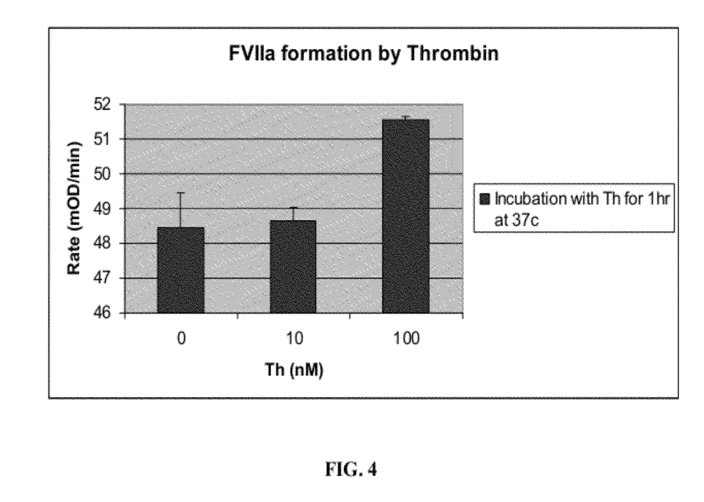

Human FVII (1 μM) was incubated with varying amounts of thrombin (0, 10, 100 nM) for 1 hour at 37° C. in HBSAC (12.5 mM HEPES pH 7.4, 100 mM NaCl, 5 mM CaCl₂, 0.1% w/v BSA, 0.05% w/v NaN₃). Hirudin was then added at a 5-fold molar excess (0, 50, 500 nM) to each reaction and incubated for 5 minutes at room temperature followed by the addition of the chromogenic substrate Chromozym-tPA (N-methylsulfonyl-D-Phe-Gly-Arg-4-nitranilide acetate) (Roche, Indianapolis, Ind.). The absorbance at 405 nm was then monitored every 15 seconds for 15 minutes to determine the rate of substrate hydrolysis. The results are shown in FIG. 4.

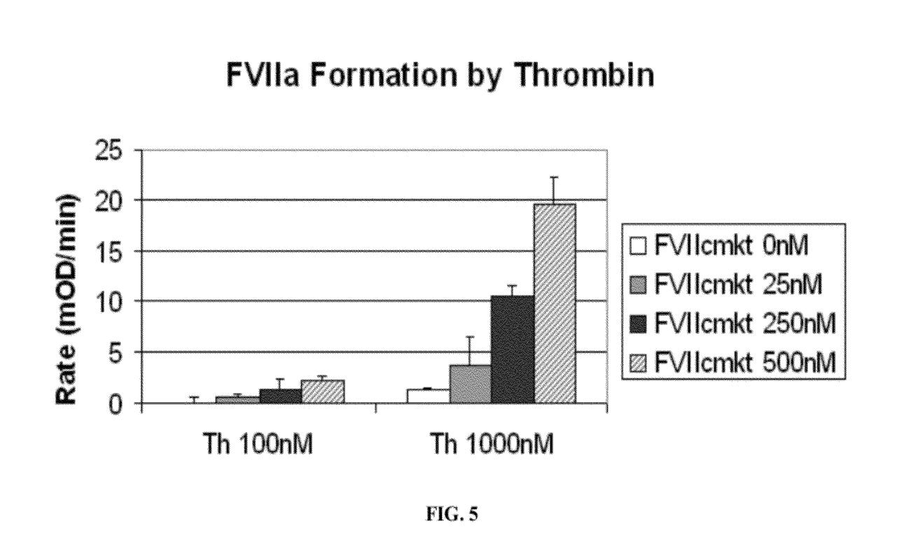

A substrate form of human FVII was also tested in which active serine protease contaminants were inhibited by treatment with a ‘Phe-Pro-Arg’ peptide based chloromethylketone (CMK) irreversible inhibitor (Haematological Technologies, Essex Junction, Vt.). When this CMK-inhibited FVII was utilized as the thrombin substrate, the background activity in the absence of thrombin was much lower and a low activation of FVII by thrombin was measured. The results are shown in FIG. 5.

In order to demonstrate the cofactor activity of the fusion proteins, the media from cells expressing the fusion protein was used with or without additional purification. Samples of FVII (with or without CMK treatment) were tested in a concentration range between 1 to 10,000 nM in the presence of a fusion protein and thrombin in a concentration range between 0.1 and 3000 nM. The assay conditions were similar to those described above for activation of FVII by thrombin alone. When FVII activation to FVIIa by thrombin is compared in the presence or absence of a fusion protein, the rate of FVII activation by thrombin is increased between 1.5 to over 10,000-fold increase under conditions where the concentration of the fusion protein ranges from between 0.1 nM to 10,000 nM.

### Example 5

### Linker Length Affects FVII Activation

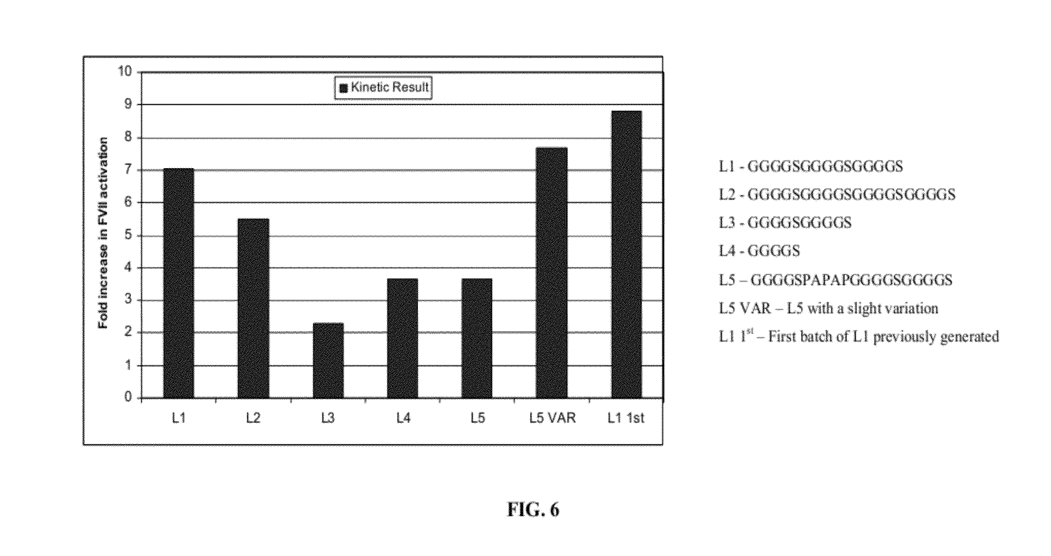

As shown in FIG. 6, variation of the linker length can affect FVII activation. L5 variant is an error in cloning that eliminated a portion of the soluble tissue factor domain. It was included as a potential control for what might happen if the affinity of tissue factor for FVII was reduced. L1 1st refers to a first prep of the L1 version of the fusion protein. This was used as a control for subsequent batches to determine how consistent, batch to batch, the fusion proteins were.

### Example 6

### Fusion Protein Enhanced Coagulation Assay

The ability of the fusion proteins to increase coagulation activity was determined using an aPTT assay in normal human plasma, FIX-deficient human plasma, or FVIII-deficient plasma. The aPPT assays with all plasma samples were run on a Electra™ 1800C automatic coagulation analyzer (Beckman Coulter, Fullerton, Calif.). Briefly, three dilutions of fusion protein samples in coagulation diluent were prepared, and 100 μL of each sample was then mixed with 100 μL of a human derived plasma and 100 μL automated aPTT reagent (rabbit brain phospholipid and micronized silica (bioMérieux, Inc., Durham, N.C.). After the addition of 100 μL 25 mM CaCl₂ solution, the time to clot formation was recorded. The time to clot was decreased by the addition of fusion protein, compared with control additions of buffer or media alone.

### Example 7

### Measurement of Circulating Fusion Protein

The circulating half-life of a fusion protein is measured in vivo using standard techniques well-known to those of ordinary skill in the art. Briefly, the respective dose of fusion protein is administered to a subject by intravenous injection, subcutaneous injection, or intradermal injection. Blood samples are taken at a number of time points after injection and the fusion protein concentration is determined by an appropriate assay. To determine the half-life, that is the time at which the concentration of fusion protein is half of the concentration of fusion protein immediately after dosing, the fusion protein concentration at the various time points is compared to the fusion protein concentration expected or measured immediately after administering the dose of fusion protein. Pharmacokinetic studies in normal mice, FIX-deficient mice, FVIII-deficient mice, rabbits, dogs, and monkeys are performed by injection of between 0.01 to 30 mg per kg of fusion protein.

An ELISA such as a sandwich ELISA, may be used to measure the circulating half-life of a fusion protein. This sandwich ELISA is based on the ability of antibody coated plates to capture the peptide FLAG-tag of the fusion protein. The amount of fusion protein captured is quantified by detection with a secondary antibody to the tissue factor component of the fusion protein.

### Example 8

### Measurement of Efficacy of Fusion Protein in Hemophilia Models

The efficacy of a fusion protein may be measured utilizing, for example, a kidney laceration model or a tail vein bleeding model. In the kidney laceration model, hemophilic mice (C57/BL6 with a disrupted FVIII gene) are anesthetized under isofluorane and weighed. The inferior vena cava is exposed and 100 uL of either saline or a fusion protein are injected using a 31 gauge needle. The needle is carefully removed and pressure applied at the site of injection for 30-45 seconds to prevent bleeding. After two minutes, the right kidney is exposed and held between the forceps along the vertical axis. Using a #15 scalpel, the kidney is cut horizontally to a depth of 3 mm. To insure a uniform depth of the lesion, the kidney is lightly held in the middle to expose equal tissue on either side of the forceps. The exposed surface of the kidney is cut to the depth of the forceps, and blood loss is quantified. Different doses of fusion protein are tested to characterize the dose response relationship of the fusion protein on kidney bleeding.

Using the tail vein bleeding model, a 200 uL disposable pipetter tip is cut 1.0 cm from its narrow end and slipped onto the tail of an anaesthetized mouse. The pipette tip is positioned towards the body of the mouse until the tail completely fills the opening and this point is marked with an indelible pen. After removal of the pipette tip, the tail is transected by incision with a fresh scalpel.

For both models, the blood is collected every 30 to 90 seconds for 15 minutes or more onto filter paper discs. The filters are then eluted in purified water for several hours of overnight. The hemoglobin derived color from lysed red blood cells is determined using a standard curve constructed from diluted citrated mouse blood and quantified using a spectrophotometer at wavelengths of 405 and 492 nm.

### Example 9

### Glycosylation

Fusion proteins containing chondroitin sulfate or similar glycosaminoglycans are analyzed by chondroitin ABC lyase digestion of the fusion protein followed by SDS-PAGE analysis (see, e.g., Lin, et al., J. Biol. Chem. 269:25021-30, 1994). Pure fusion protein or cell supernatants containing secreted fusion protein are diluted to approximately 1 to 100 ng/mL in phosphate-buffered saline with 0.05% Tween®-20 (polyoxyethylenesorbitan monolaurate) and 0.1% bovine serum albumin in duplicate. Chondroitinase ABC lysase is added to one duplicate and both samples can be incubated at 37° C. for 1 hour (Parkinson, et al., Biochem. J. 283:151-157, 1992), then compared by SDS-PAGE.

All publications and patents mentioned in the above specification are incorporated herein by reference. Various modifications and variations of the described methods of the invention will be apparent to those skilled in the art without departing from the scope and spirit of the invention.

Although the invention has been described in connection with specific embodiments, it should be understood that the invention as claimed should not be unduly limited to such specific embodiments. Indeed, various modifications of the above-described modes for carrying out the invention which are obvious to those skilled in the field of biochemistry or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

1. A fusion protein wherein said protein binds thrombin.

2. The fusion protein of claim 1, wherein said protein comprises one or more thrombin binding domains.

3. The fusion protein of claim 2, wherein said thrombin binding domain is selected from thrombomodulin thrombin binding domain, HCII thrombin binding domain, PAR1 thrombin binding domain, FVIII thrombin binding domain, OPN thrombin binding domain, HIR thrombin binding domain, FV thrombin binding domain, and FXI thrombin binding domain.

4. The fusion protein of claim 3, wherein said thrombin binding domain is selected from SEQ ID NO: 28-30 and SEQ ID NO: 32-38.

5. The fusion protein of claim 2, wherein said protein comprises one or more FVII binding domains.

6. The fusion protein of claim 5, wherein said FVII binding domain is selected from TF FVII binding domain or EPCR FVII binding domain.

7. The fusion protein of claim 6, wherein said FVII binding domain is selected from SEQ ID NO: 27 and SEQ ID NO: 31.

8. The fusion protein of claim 2, wherein said protein comprises a linker

9. The fusion protein of claim 8, wherein said linker is selected from SEQ ID NO: 2-19 and SEQ ID NO: 93-94.

10. The fusion protein of claim 2, wherein said protein comprises a secretion signal.

11. The fusion protein of claim 10, wherein said secretion signal is selected from a TF secretion signal, thrombomodulin secretion signal, EPCR secretion signal, kappa light chain secretion signal, and FXI secretion signal.

12. The fusion protein of claim 10, wherein said secretion signal is selected from SEQ ID NO: 20-26.

13. The fusion protein of claim 2, wherein said protein comprises a peptide tag.

14. The fusion protein of claim 13, wherein said peptide tag is selected from FLAG tag, c-myc tag, E-tag, and 6× His tag.

15. The fusion protein of claim 14, wherein said peptide tag is selected from SEQ ID NO: 39 and 40.

16. The fusion protein of claim 1, wherein said protein comprises one or more thrombin binding domains, one or more FVII binding domains, a linker, and a secretion signal.

17. The fusion protein of claim 16, wherein the thrombin binding domain is selected from SEQ ID NO: 28-30 and SEQ ID NO: 32-38, the FVII binding domain is selected from SEQ ID NO: 27 and SEQ ID NO: 31, the linker selected from SEQ ID NO: 2-19, and the secretion signal selected from SEQ ID NO: 20-26.

18. The fusion protein of claim 17, wherein said protein is selected from SEQ ID NO: 41, 43, 45, 47, 49, and 51-84.

19. The fusion protein of claim 18, wherein said protein comprise a peptide tag.

20. The fusion protein of claim 19, wherein said peptide tag is selected from FLAG tag, c-myc tag, E-tag, and 6× His tag.

21. The fusion protein of claim 20, wherein said peptide tag is selected from SEQ ID NO: 39 and 40.

22. A polynucleotide wherein said polynucleotide encodes the fusion protein of claim 1.

23. The polynucleotide of claim 22, wherein said polynucleotide is selected from SEQ ID NO:
42, 44, 46, 48, and 50.

24. A vector wherein said vector comprises the polynucleotide sequence of claim 22.

25. A host cell wherein said cell is transfected with the vector of claim 24.

26. A pharmaceutical composition comprising the fusion protein of claim 1.

27. A method of treating a bleeding disorder comprising administering to a subject in need thereof an effective amount of the pharmaceutical composition of claim 26.

28. The method of claim 27, wherein the pharmaceutical composition is administered prophylactically.

29. A method of treating hemophilia comprising administering to a subject in need thereof an effective amount of the pharmaceutical composition of claim 26.

30. The method of claim 29, wherein the pharmaceutical composition is administered prophylactically.


SEQUENCE LISTING (94 sequences)

SEQ ID NO: 1 - protein, 8 aa, Artificial Sequence
  Cleavage site of FVII
PQGRIVGG

SEQ ID NO: 2 - protein, 8 aa, Artificial Sequence
  Linker Sequence
GSIGGGIS

SEQ ID NO: 3 - protein, 25 aa, Artificial Sequence
  Linker Sequence
GSIGGGGSGGGGSGGGGSGGGGSIS

SEQ ID NO: 4 - protein, 30 aa, Artificial Sequence
  Linker Sequence
GSIGGGGSGGGGSGGGGSGGGGSGGGGSIS

SEQ ID NO: 5 - protein, 30 aa, Artificial Sequence
  Linker sequence
GSIGSGGGGSGGGGSGGGGSGGGGSGGGIS

SEQ ID NO: 6 - protein, 24 aa, Artificial Sequence
  Linker sequence
GSIGSGGGGSGGGGSGGGGSGGIS

SEQ ID NO: 7 - protein, 10 aa, Artificial Sequence
  Linker sequence
GGGGSGGGGS

SEQ ID NO: 8 - protein, 15 aa, Artificial Sequence
  Linker sequence
GGGGSGGGGSGGGGS

SEQ ID NO: 9 - protein, 20 aa, Artificial Sequence
  Linker sequence
GGGGSGGGGSGGGGSGGGGS

SEQ ID NO: 10 - protein, 25 aa, Artificial Sequence
  Linker sequence
GGGGSGGGGSGGGGSGGGGSGGGGS

SEQ ID NO: 11 - protein, 30 aa, Artificial Sequence
  Linker sequence
GGGGSGGGGSGGGGSGGGGSGGGGSGGGGS

SEQ ID NO: 12 - protein, 35 aa, Artificial Sequence
  Linker sequence
GGGGSGGGGSGGGGSGGGGSGGGGSGGGGSGGGGS

SEQ ID NO: 13 - protein, 30 aa, Artificial Sequence
  Linker sequence
GGGGSGGGGSPAPAPGGGGSGGGGSGGGGS

SEQ ID NO: 14 - protein, 30 aa, Artificial Sequence
  Linker sequence
GGGGSGGGGSGGGGSPAPAPGGGGSGGGGS

SEQ ID NO: 15 - protein, 30 aa, Artificial Sequence
  Linker sequence
GGGGSPAPAPGGGGSGGGGSPAPAPGGGGS

SEQ ID NO: 16 - protein, 30 aa, Artificial Sequence
  Linker sequence
GSGGSGSGGSGSGGSGSGGSGSGGSGSGGS

SEQ ID NO: 17 - protein, 30 aa, Artificial Sequence
  Linker sequence
GSGGSGSGGSGGPAPAPGGSGSGGSGSGGS

SEQ ID NO: 18 - protein, 30 aa, Artificial Sequence
  Linker sequence
GGGGSGGGGAEAAAKEAAAKAGGGSGGGGS

SEQ ID NO: 19 - protein, 35 aa, Artificial Sequence
  Linker Sequence/Thrombomodulin O-link domain
DSGKVDGGDSGSGEPPPSPTPGSTLTPPAVGLVHS

SEQ ID NO: 20 - protein, 32 aa, Artificial Sequence
  Modified TF secretion signal sequence
METPAWPRVPRPGTAVARTLLLGWVFAQVAGA

SEQ ID NO: 21 - protein, 32 aa, Artificial Sequence
  TF secretion signal
METPAWPRVPRPETAVARTLLLGWVFAQVAGA

SEQ ID NO: 22 - protein, 18 aa, Artificial Sequence
  Modified thrombomodulin secretion signal sequence
MLGVLVLGALALAGLVFP

SEQ ID NO: 23 - protein, 18 aa, Artificial Sequence
  Thrombomodulin secretion signal
MLGVLVLGALALAGLGFP

SEQ ID NO: 24 - protein, 15 aa, Artificial Sequence
  Endothelial protein C receptor secretion signal sequence
MLTTLLPILLLSGWA

SEQ ID NO: 25 - protein, 23 aa, Artificial Sequence
  Kappa light chain secretion signal
METDTLLLWVLLLWVPGSTGDAA

SEQ ID NO: 26 - protein, 18 aa, Artificial Sequence
  Factor XI Apple-1 secretion signal sequence
MIFLYQVVHFILFTSVSG

SEQ ID NO: 27 - protein, 219 aa, Artificial Sequence
  TF FVII-binding domain
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 28 - protein, 92 aa, Artificial Sequence
  TMcE56 ABE thrombin binding domain
VCAEGFAPIPGEPHRCQLFCNQTACPADCDPNTQASCECPEGYILDDGFICTDIDECENGGFCSGVCHNLPGTFECICGPDSALAGQIGTDC

SEQ ID NO: 29 - protein, 102 aa, Artificial Sequence
  TMcE56-B
AVCAEGFAPIPHEPHRCQMFCNQTACPADCDPNTQASCECPEGYILDDGFICTDIDECENGGFCSGVCHNLPGTFECICGPDSALVRHIGTDCDSGKVDGGD

SEQ ID NO: 30 - protein, 93 aa, Artificial Sequence
  TMcE56-C
AVCAEGFAPIPHEPHRCQMFCNQTACPADCDPNTQASCECPEGYILDDGFICTDIDECENGGFCSGVCHNLPGTFECICGPDSALVRHIGTDC

SEQ ID NO: 31 - protein, 195 aa, Artificial Sequence
  EPCR FVII-binding domain
FCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTS

SEQ ID NO: 32 - protein, 25 aa, Artificial Sequence
  HCII ABE thrombin binding domain
PEGEEDDDYLDLEKIFSEDDDYIDI

SEQ ID NO: 33 - protein, 21 aa, Artificial Sequence
  PAR1 ABE thrombin binding domain
NDKYEPFWEDEEKNESGLTEY

SEQ ID NO: 34 - protein, 30 aa, Artificial Sequence
  FVIII ABE thrombin binding domain
NTGDYYEDSYEDISAYLLSKNNAIEPRSFS

SEQ ID NO: 35 - protein, 20 aa, Artificial Sequence
  OPN ABE thrombin binding domain
DIQYPDATDEDITSHMESEE

SEQ ID NO: 36 - protein, 14 aa, Artificial Sequence
  HIR ABE thrombin binding domain
NNGDFEEIPEEYLQ

SEQ ID NO: 37 - protein, 56 aa, Artificial Sequence
  FV ABE thrombin binding domain
PDDDEDSYEIFEPPESTVMATRKMHDRLEPEDEESDADYDYQNRLAAALGIRSFRN

SEQ ID NO: 38 - protein, 91 aa, Artificial Sequence
  FIX-Apple1 thrombin binding domain
ECVTQLLKDTCFEGGDITTVFTPSAKYCQVVCTYHPRCLLFTFTAESPSEDPTRWFTCVLKDSVTETLPRVNRTAAISGYSFKQCSHQISA

SEQ ID NO: 39 - protein, 18 aa, Artificial Sequence
  E-tag sequence
AAAGAPVPYPDPLEPRAA

SEQ ID NO: 40 - protein, 11 aa, Artificial Sequence
  FLAG-tag sequence
AAADYKDDDDK

SEQ ID NO: 41 - protein, 351 aa, Artificial Sequence
  sTF-TMcE56-A
METPAWPRVPRPGTAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREGSIGGGISVCAEGFAPIPGEPHRCQLFCNQTACPADCDPNTQASCECPEGYILDDGFICTDIDECENGGFCSGVCHNLPGTFECICGPDSALAGQIGTDC

SEQ ID NO: 42 - DNA, 1107 bp, Artificial Sequence
  sTF-TMcE56-A
atggagacccctgcctggccccgggtcccgcgccccgggaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaggatcgattgggggcgggatatccgtctgcgccgagggcttcgcgcccattcccggcgagccgcacaggtgccagctgttttgcaaccagaccgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgccggccagattggcaccgactgtgcggccgcaggagctccggtgccgtatccggatccgctggaaccgcgtgccgca

SEQ ID NO: 43 - protein, 368 aa, Artificial Sequence
  sTF-TMcE56-B
METPAWPRVPRPGTAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREGSIGGGGSGGGGSGGGGSGGGGSISVCAEGFAPIPGEPHRCQLFCNQTACPADCDPNTQASCECPEGYILDDGFICTDIDECENGGFCSGVCHNLPGTFECICGPDSALAGQIGTDC

SEQ ID NO: 44 - DNA, 1158 bp, Artificial Sequence
  sTF-TMcE56-B
atggagacccctgcctggccccgggtcccgcgccccgggaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaggatcgattggcggtggtggctccggtggcggtggtagtggcggtggtggctccggcggtggtggctcgatatccgtctgcgccgagggcttcgcgcccattcccggcgagccgcacaggtgccagctgttttgcaaccagaccgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgccggccagattggcaccgactgtgcggccgcaggagctccggtgccgtatccggatccgctggaaccgcgtgccgca

SEQ ID NO: 45 - protein, 373 aa, Artificial Sequence
  sTF-TMcE56-C
METPAWPRVPRPGTAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREGSIGGGGSGGGGSGGGGSGGGGSGGGGSISVCAEGFAPIPGEPHRCQLFCNQTACPADCDPNTQASCECPEGYILDDGFICTDIDECENGGFCSGVCHNLPGTFECICGPDSALAGQIGTDC

SEQ ID NO: 46 - DNA, 1173 bp, Artificial Sequence
  sTF-TMcE56-C
atggagacccctgcctggccccgggtcccgcgccccgggaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaggatcgattggcggtggtggctccggcggtggtggcagcggtggcggtggtagtggcggtggtggctccggcggtggtggctcgatatccgtctgcgccgagggcttcgcgcccattcccggcgagccgcacaggtgccagctgttttgcaaccagaccgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgccggccagattggcaccgactgtgcggccgcaggagctccggtgccgtatccggatccgctggaaccgcgtgccgca

SEQ ID NO: 47 - protein, 373 aa, Artificial Sequence
  sTF-TMcE56-D
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREGSIGSGGGGSGGGGSGGGGSGGGGSGGGISVCAEGFAPIPHEPHRCQMFCNQTACPADCDPNTQASCECPEGYILDDGFICTDIDECENGGFCSGVCHNLPGTFECICGPDSALVRHIGTDC

SEQ ID NO: 48 - DNA, 1152 bp, Artificial Sequence
  sTF-TMcE56-D
atggagacgcctgcttggccacgggtacccagacctgagacagccgtggccagaacattgttgctcggttgggtcttcgctcaggtcgctggcgcctccgggaccactaacactgtggcagcctacaacctgacttggaaatcaacaaattttaagaccatcctggaatgggagccaaagcctgtcaatcaggtctacactgttcagatctccaccaaaagcggagactggaagtcaaaatgcttctataccaccgatacagagtgtgacctgaccgatgagattgtcaaggatgttaagcaaacataccttgcacgcgtgttcagttaccctgctgggaacgtggaaagcaccggaagcgctggagagccactgtatgagaatagtcccgagtttaccccatacctggagacaaatcttggtcagcctacgattcaatccttcgagcaggttggcaccaaggtcaatgtgaccgtggaagacgagcgcacactggtcagacggaacaatacctttctttccctccgcgatgtgtttggcaaagacctgatctatacactgtactactggaagtcctcatccagcggaaaaaaaaccgctaaaaccaatactaatgagttcctgatagatgtagataaaggggaaaattactgctttagcgttcaggccgtaatccctagccgcacagttaatagaaagtccaccgactcacccgtggagtgcatggggcaggaaaaaggcgaatttcgcgagggatcgattggctccggaggtggtggaagcgggggtgggggttccggtggtggaggttctgggggcgggggaagcggaggagggatatccgtctgtgcagaaggatttgcacccataccacatgagccccaccggtgccaaatgttctgcaaccagactgcctgtccagccgactgtgaccccaacactcaggcctcttgtgaatgtccagaagggtatatcttggacgatggtttcatctgcaccgacatcgacgaatgtgagaatggagggttttgcagcggagtgtgccacaaccttcctggtacttttgaatgcatttgtggccccgatagtgcccttgtccgacacatcggaactgattgcgcggctgccgactacaaagatgacgatgacaag

SEQ ID NO: 49 - protein, 363 aa, Artificial Sequence
  TMcE56-sTF
MLGVLVLGALALAGLVFPAVCAEGFAPIPHEPHRCQMFCNQTACPADCDPNTQASCECPEGYILDDGFICTDIDECENGGFCSGVCHNLPGTFECICGPDSALVRHIGTDCDSGKVDGGDGSIGSGGGGSGGGGSGGGGSGGISSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 50 - DNA, 1122 bp, Artificial Sequence
  TMcE56-sTF
atgcttggagttctcgtacttggcgccctcgcgctggcaggccttgtgtttcccgccgtgtgcgctgaaggattcgctcctatcccgcacgaacctcaccgatgtcagatgttctgcaatcagaccgcttgtcccgctgactgcgaccccaatacccaggccagctgcgagtgtcctgaaggctacattcttgatgacggctttatctgcaccgacattgacgaatgtgagaatggggggttttgctccggcgtgtgccacaatctgcccggcaccttcgagtgtatctgcggccccgattcagcgttggtgcggcacataggaactgactgtgacagcggcaaggtggacgggggagatggatcgattggttctggcggaggcggtagcggcggcgggggtagtgggggcggcgggtctggcgggatatccagtggcaccactaacacggtcgcagcctacaacttgacctggaagtccacaaacttcaaaacgattctggagtgggaacccaagccggtcaaccaagtgtacacagtgcagatcagcacaaagagtggcgattggaaatccaaatgtttctacaccactgacaccgagtgcgatctgaccgacgagattgtcaaggacgtcaaacagacatacctcgcccgcgttttctcctaccctgctgggaacgtggagtctaccggctctgcaggcgaaccactctatgaaaactctcctgagtttactccctatctggagaccaatctcggccagcctaccatacagtcattcgaacaggtgggaacaaaggtgaatgttaccgtagaagacgaacggaccctggtgcgccggaataataccttcctctccctgagagacgtcttcggaaaggacctcatctacacactgtattactggaagtcaagctcttccggaaagaaaactgcaaagactaacacaaatgagttcctcatagacgtggataaaggggagaattattgcttctccgtgcaggccgtgatcccatcaagaacagttaatagaaagtctacagattcccccgttgagtgtatggggcaggaaaaaggagagttccgagaggcagcagcggactacaaggacgacgacgacaaa

SEQ ID NO: 51 - protein, 408 aa, Artificial Sequence
  sTF-TMcE56 O-link
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREGSIGSGGGGSGGGGSGGGGSGGGGSGGGISVCAEGFAPIPHEPHRCQMFCNQTACPADCDPNTQASCECPEGYILDDGFICTDIDECENGGFCSGVCHNLPGTFECICGPDSALVRHIGTDCDSGKVDGGDSGSGEPPPSPTPGSTLTPPAVGLVHS

SEQ ID NO: 52 - protein, 365 aa, Artificial Sequence
  TMcE56-OlinkCS-sTF
MLGVLVLGALALAGLVFPAVCAEGFAPIPHEPHRCQMFCNQTACPADCDPNTQASCECPEGYILDDGFICTDIDECENGGFCSGVCHNLPGTFECICGPDSALVRHIGTDCDSGKVDGGDSGSGEPPPSPTPGSTLTPPAVGLVHSSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 53 - protein, 332 aa, Artificial Sequence
  sEPCR-TMcE56
MLTTLLPILLLSGWAFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTSGSIGSGGGGSGGGGSGGGGSGGGGSGGGISVCAEGFAPIPHEPHRCQMFCNQTACPADCDPNTQASCECPEGYILDDGFICTDIDECENGGFCSGVCHNLPGTFECICGPDSALVRHIGTDC

SEQ ID NO: 54 - protein, 339 aa, Artificial Sequence [sequence omitted]

SEQ ID NO: 55 - protein, 367 aa, Artificial Sequence [sequence omitted]

SEQ ID NO: 56 - protein, 341 aa, Artificial Sequence [sequence omitted]

SEQ ID NO: 57 - protein, 306 aa, Artificial Sequence
  HCIIABE-sTF
METPAWPRVPRPETAVARTLLLGWVFAQVAGAPEGEEDDDYLDLEKIFSEDDDYIDIGSIGSGGGGSGGGGSGGGGSGGGGSGGGISSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 58 - protein, 306 aa, Artificial Sequence
  sTF-HCIIABE
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREGSIGSGGGGSGGGGSGGGGSGGGGSGGGISPEGEEDDDYLDLEKIFSEDDDYIDI

SEQ ID NO: 59 - protein, 302 aa, Artificial Sequence
  PAR1ABE-sTF
METPAWPRVPRPETAVARTLLLGWVFAQVAGANDKYEPFWEDEEKNESGLTEYGSIGSGGGGSGGGGSGGGGSGGGGSGGGISSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 60 - protein, 302 aa, Artificial Sequence
  sTF-PAR1ABE
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREGSIGSGGGGSGGGGSGGGGSGGGGSGGGISNDKYEPFWEDEEKNESGLTEY

SEQ ID NO: 61 - protein, 311 aa, Artificial Sequence
  FVIIIABE-sTF
METPAWPRVPRPETAVARTLLLGWVFAQVAGANTGDYYEDSYEDISAYLLSKNNAIEPRSFSGSIGSGGGGSGGGGSGGGGSGGGGSGGGISSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 62 - protein, 311 aa, Artificial Sequence
  sTF-FVIIIABE
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREGSIGSGGGGSGGGGSGGGGSGGGGSGGGISNTGDYYEDSYEDISAYLLSKNNAIEPRSFS

SEQ ID NO: 63 - protein, 301 aa, Artificial Sequence
  OPNABE-sTF
METPAWPRVPRPETAVARTLLLGWVFAQVAGADIQYPDATDEDITSHMESEEGSIGSGGGGSGGGGSGGGGSGGGGSGGGISSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 64 - protein, 301 aa, Artificial Sequence
  sTF-OPNABE
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREGSIGSGGGGSGGGGSGGGGSGGGGSGGGISDIQYPDATDEDITSHMESEE

SEQ ID NO: 65 - protein, 295 aa, Artificial Sequence
  HIRABE-sTF
METPAWPRVPRPETAVARTLLLGWVFAQVAGANNGDFEEIPEEYLQGSIGSGGGGSGGGGSGGGGSGGGGSGGGISSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 66 - protein, 295 aa, Artificial Sequence
  sTF-HIRABE
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREGSIGSGGGGSGGGGSGGGGSGGGGSGGGISNNGDFEEIPEEYLQ

SEQ ID NO: 67 - protein, 337 aa, Artificial Sequence
  FVABE-sTF
METPAWPRVPRPETAVARTLLLGWVFAQVAGAPDDDEDSYEIFEPPESTVMATRKMHDRLEPEDEESDADYDYQNRLAAALGIRSFRNGSIGSGGGGSGGGGSGGGGSGGGGSGGGISSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 68 - protein, 337 aa, Artificial Sequence [sequence omitted]

SEQ ID NO: 69 - protein, 358 aa, Artificial Sequence
  Apple1-sTF
MIFLYQVVHFILFTSVSGECVTQLLKDTCFEGGDITTVFTPSAKYCQVVCTYHPRCLLFTFTAESPSEDPTRWFTCVLKDSVTETLPRVNRTAAISGYSFKQCSHQISAGSIGSGGGGSGGGGSGGGGSGGGGSGGGISSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 70 - protein, 372 aa, Artificial Sequence [sequence omitted]

SEQ ID NO: 71 - protein, 282 aa, Artificial Sequence
  HCIIABE-sEPCR
METPAWPRVPRPETAVARTLLLGWVFAQVAGAPEGEEDDDYLDLEKIFSEDDDYIDIGSIGSGGGGSGGGGSGGGGSGGGGSGGGISFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTS

SEQ ID NO: 72 - protein, 265 aa, Artificial Sequence
  sEPCR-HCIIABE
MLTTLLPILLLSGWAFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTSGSIGSGGGGSGGGGSGGGGSGGGGSGGGISPEGEEDDDYLDLEKIFSEDDDYIDI

SEQ ID NO: 73 - protein, 278 aa, Artificial Sequence
  PAR1-sEPCR
METPAWPRVPRPETAVARTLLLGWVFAQVAGANDKYEPFWEDEEKNESGLTEYGSIGSGGGGSGGGGSGGGGSGGGGSGGGISFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTS

SEQ ID NO: 74 - protein, 261 aa, Artificial Sequence
  sEPCR-PAR1
MLTTLLPILLLSGWAFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTSGSIGSGGGGSGGGGSGGGGSGGGGSGGGISNDKYEPFWEDEEKNESGLTEY

SEQ ID NO: 75 - protein, 287 aa, Artificial Sequence
  FVIIIABE-sEPCR
METPAWPRVPRPETAVARTLLLGWVFAQVAGANTGDYYEDSYEDISAYLLSKNNAIEPRSFSGSIGSGGGGSGGGGSGGGGSGGGGSGGGISFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTS

SEQ ID NO: 76 - protein, 270 aa, Artificial Sequence
  sEPCR-FVIIIABE
MLTTLLPILLLSGWAFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTSGSIGSGGGGSGGGGSGGGGSGGGGSGGGISNTGDYYEDSYEDISAYLLSKNNAIEPRSFS

SEQ ID NO: 77 - protein, 277 aa, Artificial Sequence
  OPN-sEPCR
METPAWPRVPRPETAVARTLLLGWVFAQVAGADIQYPDATDEDITSHMESEEGSIGSGGGGSGGGGSGGGGSGGGGSGGGISFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTS

SEQ ID NO: 78 - protein, 260 aa, Artificial Sequence
  sEPCR-OPN
MLTTLLPILLLSGWAFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTSGSIGSGGGGSGGGGSGGGGSGGGGSGGGISDIQYPDATDEDITSHMESEE

SEQ ID NO: 79 - protein, 271 aa, Artificial Sequence
  HIR-sEPCR
METPAWPRVPRPETAVARTLLLGWVFAQVAGANNGDFEEIPEEYLQGSIGSGGGGSGGGGSGGGGSGGGGSGGGISFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTS

SEQ ID NO: 80 - protein, 254 aa, Artificial Sequence
  sEPCR-HIR
MLTTLLPILLLSGWAFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTSGSIGSGGGGSGGGGSGGGGSGGGGSGGGISNNGDFEEIPEEYLQ

SEQ ID NO: 81 - protein, 313 aa, Artificial Sequence
  FVABE-sEPCR
METPAWPRVPRPETAVARTLLLGWVFAQVAGAPDDDEDSYEIFEPPESTVMATRKMHDRLEPEDEESDADYDYQNRLAAALGIRSFRNGSIGSGGGGSGGGGSGGGGSGGGGSGGGISFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTS

SEQ ID NO: 82 - protein, 296 aa, Artificial Sequence
  sEPCR-FVABE
MLTTLLPILLLSGWAFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTSGSIGSGGGGSGGGGSGGGGSGGGGSGGGISPDDDEDSYEIFEPPESTVMATRKMHDRLEPEDEESDADYDYQNRLAAALGIRSFRN

SEQ ID NO: 83 - protein, 334 aa, Artificial Sequence [sequence omitted]

SEQ ID NO: 84 - protein, 331 aa, Artificial Sequence
  sEPCR-Apple1
MLTTLLPILLLSGWAFCSQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTSGSIGSGGGGSGGGGSGGGGSGGGGSGGGISECVTQLLKDTCFEGGDITTVFTPSAKYCQVVCTYHPRCLLFTFTAESPSEDPTRWFTCVLKDSVTETLPRVNRTAAISGYSFKQCSHQISA

SEQ ID NO: 85 - DNA, 40 bp, Artificial Sequence
  sTF primer
gcgcccaagcttgcgatggagacccctgcctggccccggg

SEQ ID NO: 86 - DNA, 55 bp, Artificial Sequence
  sTF primer
gacggatatcccgcccccaatcgatccttctctgaattcccctttctcctggccc

SEQ ID NO: 87 - DNA, 42 bp, Artificial Sequence
  Soluble thrombomodulin domain primer
ggcgggatatccgtctgcgccgagggcttcgcgcccattccc

SEQ ID NO: 88 - DNA, 42 bp, Artificial Sequence
  Soluble thrombomodulin domain primer
gccgctcgagcggtcatgcggcacgcggttccagcggatccg

SEQ ID NO: 89 - DNA, 67 bp, Artificial Sequence
  Linker oligo
cgattggcggtggtggctccggtggcggtggtagtggcggtggtggctccggcggtggtggctcgat

SEQ ID NO: 90 - DNA, 65 bp, Artificial Sequence
  Linker oligo
atcgagccaccaccgccggagccaccaccgccactaccaccgccaccggagccaccaccgccaat

SEQ ID NO: 91 - DNA, 82 bp, Artificial Sequence
  Linker oligo
cgattggcggtggtggctccggcggtggtggcagcggtggcggtggtagtggcggtggtggctccggcggtggtggctcgat

SEQ ID NO: 92 - DNA, 80 bp, Artificial Sequence
  Linker oligo
atcgagccaccaccgccggagccaccaccgccactaccaccgccaccgctgccaccaccgccggagccaccaccgccaat

SEQ ID NO: 93 - protein, 5 aa, Artificial
  Linker
GGGGS

SEQ ID NO: 94 - protein, 20 aa, Artificial
  Linker
GGGGSPAPAPGGGGSGGGGS